(19) United States
(12) Patent Application Publication
(10) Pub. No.: US 2005/0142137 A1
(43) Pub. Date: Jun. 30, 2005

(54) Antibodies against secretoryleukocyte protease inhibitor

(75) Inventors: Michael Gallo, North Vancouver (CA); Daniel Chui, Burnaby (CA); Haihong Zhong, Guilford, CT (US); Gulshan Ara, Northford, CT (US); William J. LaRochelle, Madison, CT (US)

Correspondence Address: GEORGE YAHWAK ESQ., 555 LONG WHARF DRIVE, 9TH FLOOR, NEW HAVEN, CT, 06511, US

(73) Assignee: CuraGen Corporation, New Haven, CT (US)

(21) Appl. No.: 10/984,960

(22) Filed: Nov. 8, 2004

Related U.S. Application Data
(60) Provisional application No. 60/518,275, filed on Nov. 7, 2003.

(51) Int. Cl. A61K 39/395; C07K 16/40

(52) U.S. Cl. 424/146.1; 530/388.26

(57) ABSTRACT

In accordance with the present invention, there are provided fully human monoclonal antibodies against secretory leukocyte protease inhibitor (SLPI) and antigen binding fragments thereof. Nucleotide sequences encoding and amino acid sequences comprising heavy and light chain immunoglobulin molecules are provided. The invention provides antibodies and antigen-binding fragments thereof specifically bind to SLPI; act to neutralize SLPI activity; and can modulate SLPI effects.

### RELATED APPLICATION

This Application claims the benefit of priority from U.S. Provisional Application, Ser. No. 60/518,275 filed Nov. 7, 2003 the content of which are incorporated herein in its entirety.

### FIELD OF THE INVENTION

The invention disclosed herein is related to antibodies with specificity to Secretory leukocyte protease inhibitor (SLPI), and uses of such antibodies. In particular, there are provided fully human monoclonal antibodies that specifically bind to SLPI. Nucleotide sequences encoding, and amino acid sequences comprising, heavy and light chain immunoglobulin molecules, particularly sequences corresponding to contiguous heavy and light chain sequences spanning the framework regions and/or complementarity determining regions (CDRs), specifically from FR1 through FR4 or CDR1 through CDR3, are provided. Hybridomas or other cell lines expressing such immunoglobulin molecules and monoclonal antibodies are also provided.

### BACKGROUND OF THE INVENTION

Secretory Leukocyte Protease Inhibitor

Secretory leukocyte protease inhibitor (SLPI), also known as human seminal inhibitor-I (HUSI-I) (Seemuller U., et al. 1986 FEBS Lett. 199:43-48) and anti-leukoprotease (ALP-1), is a 12 kDa member of the kazal-type serine protease inhibitor family. Originally shown to be present in seminal fluid (Fink E., et al. 1971, Hoppe-Seyler's Z Physiol Chem 352: 1591-1594), cervical mucus (Wallner O., et al. 1974, Hoppe-Seyler's Z Physiol Chem 355: 709-715), bronchial secretions (Ohlsson K, et al. 1977 Hoppe-Seyler's Z Physiol Chem 358: 583-589) and parotid secretions (Ohlsson M., et al. 1983 Hoppe-Seyler's Z Physiol Chem 364: 1323-1328) as well as in human serum (Fryksmark U, et al. 1981 Hoppe Seyler's Z Physiol Chem 362: 1273-1277), this inhibitor was shown to be identical to antileukoprotease and its metabolites by immunoreactivity (Fryksmar; Ohlsson K) or amino acid sequence analysis (Thompson R. C., et al. 1986, Proc Natl Acad Sci USA 83: 6692-6696; Seemuller 1986).

SLPI acts to protects against neutrophil proteases during inflammatory responses (McElvaney N G., et al 1992 J. Clin. Invest. 90:1296-1301; Song X., et al 1999 J. Exp Med 190:535-542; Lentsch A B., et al. 1999 Gasroenterology 117:953-961; Gipson T S., et al 1999 J. Immuno. 162: 3653-362) as well as to promote wound healing (Ashcroft G S., 2000 Nat. Med 6: 1147-1153), cell proliferation (Zhang D., et al. 2002 J. Biol. Chem. 277:29999-30009), inhibit HIV infection (McNeely T B et al. 1997 Blood 90:1141-1149) and NF-KB activation (Lentsch A B., et al. 1999 Am. J. Pathol 154:239-247) lyse bacteria (Hiemstra P S., et al. 1996 Infect. Immun. 64: 4520-4524) and modulate macrophage functions (Zhang Y., et al. 1997 J. Clin. Invest 99:894-900).

SLPI has been shown to be expressed in respiratory epithelium (Kramps J A et al Pulmonary Emphsema and Proteolysis. Taylor J C & Mittman, C. (eds) Academic Press: New York, 1987, pp325-329; Franken et al, J Histochem Cytochem 37:493498, 1989; de Water R., et al. Am Rev Resp Dis 133: 882-890, 1986) and also in a variety of cancers including lung, breast, oropharyngeal, bladder, ovarian, endometrial and colorectal (Garver R I., et al, Gene Therapy 1:46-50, 1994).

SLPI expression has been correlated with tumor progression (Hough C D et al. 2000 Can Res. 60:6281-6287; Hough C D., et al. 2001 Cancer Res. 1:3869-3876; Shigemasa K., et al. 2001 Int. J. Gynecol. Cancer 11:454-461; Ameshima S., et al. 2000 Cancer 89:1448-1456, Morita M., et al. 1999 Ad. Enzyme. Regul. 39:341-355) perhaps through SLPI protease inhibitory activity (Devoogdt N., et al. 2003 PNAS 100:5778-5782) or promotion of cell proliferation (Zhang, D., et al., 2002 JBC).

Antibodies

The specificity of monoclonal antibodies have made them attractive agents for targeting cancer in vivo with the hopes of irradicating disease while sparing normal tissue. The approach, which initially utilized mouse monoclonal antibodies has encountered limitations to potential effectiveness such as immunogenicity; inefficient effector functions and short half-life in vivo. Technologies were developed for: chimeric antibodies which sought to utilize the antigen binding variable domains of mouse monoclonal antibodies combined with the constant regions of human antibodies (Boulianne, et al. 1984 Nature 312:643-646; Morrison et al, 1984 PNAS USA 81:6851-6855); humanized antibodies which grafted antigen binding complementary determining regions (CDRS) from mouse antibodies to human immunoglobulin (Jones, et al, 1986 Nature 321: 522-525; Riechmann, et al, 1988 Nature 332:323-327; Verhoeyen, et al, 1988 Science 239:1534-1536; Vaughan, et al, 1998 Nature Biotechnol. 16:535-539); and phage display libraries of single chain scFVs or Fab fragments of antibodies (de Haard, et al, 1999 J Biol. Chem. 274: 18218-18230; Knappik, et al, 2000 J. Mol. Biol. 296:57-86; Sheets, et al, 1998 PNAS USA 95:6157-6162; Vaughan, et al, 1994 Nature Biotechnol 14:309-314, 1996; Griffiths et al EMBO J. 13:3245-3260). Additionally, transgenic animals having human immunoglobulin genes and nonfunctional endogenous genes have been developed for immunization and production of fully human monoclonal antibodies (Fishwwild, et al, 1996 Nature Biotechnol 14:845-851; Mendez, et al, 1997 Nature Genet. 15:146-156; Nicholson, et al, 1999 J. Immunol 163, 6898-6906).

Recombinant technologies are being utilized and continue to develop seeking further improvements upon antibody molecules with the goal of enhancing in vivo efficacy. Such technologies provide, for example, for optimizing molecular size, affinity, specificity, valency, effector functions, direct and indirect arming, combination therapy, and various prodrug approaches.

### SUMMARY OF THE INVENTION

The current invention provides antibodies that specifically bind Secretory leukocyte protease inhibitor (SLPI). Further, antibodies that modulate the activity of SLPI are provided. The invention provides anti-SLPI human monoclonal antibodies, variants and derivatives thereof as well as antigen binding fragments thereof. Further, anti-SLPI human monoclonal antibodies variants and derivatives thereof and antigen binding fragments thereof that modulate the activity of SLPI are provided. Provided are anti-SLPI human monoclonal antibodies variants and derivatives thereof as well as antigen binding fragments thereof that are capable of neutralizing SLPI activity.

The invention provides preferred somatic recombinations of human antibody gene segments to provide specificity for SLPI and genetically engineered anti-SLPI antibody variants and derivatives that originate from these gene segments. In addition, the current invention provides multiple affinity matured human antibodies with binding specificity for SLPI.

Amino acid sequences for anti-SLPI human monoclonal antibodies of the invention and nucleic acid sequences encoding them are provided.

Compositions comprising human anti-SLPI antibodies, including therapeutic compositions comprising same, and methods of use are provided.

Additional aspects of the disclosure will be set forth in part in the description which follows, and in part will be obvious from the description, or may be learned by practicing the invention. The invention is set forth and particularly pointed out in the appended claims, and the present disclosure should not be construed as limiting the scope of the claims in any way. The following detailed description includes exemplary representations of various embodiments of the invention, which are not restrictive of the invention, as claimed. The accompanying figures constitute a part of this specification and, together with the description, serve only to illustrate various embodiments and not limit the invention. Citation of references is not an admission that these references are prior art to the invention.

### BRIEF DESCRIPTION OF THE FIGURES

FIG. 1: Shows the graphed results of an elastase inhibition assay in which antibodies of the invention inhibit the enzyme activity of elastase in a dose dependent manner. See Example 9 for more details.

FIG. 2: Shows the graphed results of a Cathepsin G inhibition assay in which antibodies of the current invention inhibit the enzyme activity of cathepsin G in a dose dependent manner. See Example 10.for more details.

FIG. 3: Is an image of a Western blot showing specific reactivity of the anti-SLPI antibodies of the current invention with conditioned cultured media derived from various mammalian tumor derived cell lines. See Example 12 for more details.

FIG. 4: Shows the effect of SLPI-neutralizing antibody on ovarian carcinoma cells. (A) depicts the stimulation of OVCAR-3 cell proliferation by exogenously added SLPI in a clonogenic assay. The number of colonies on each plate was counted and raw data was normalized to untreated sample and presented as % of survival. (B) depicts the inhibition of OVCAR-3 cell proliferation by antibody 42C1, with or without exogenous SLPI in a clonogenic assay. Irrelevant IgG and exogenous SLPI (◯); Irrelevant IgG (▪); Anti-SLPI antibody and exogenous SLPI (Δ); Anti-SLPI antibody (♦). See Example 15 for more details.

FIG. 5: SLPI inhibition of elastase activity (A) SLPI inhibition of elastase activity is reversed by addition of anti-SLPI antibody 42C1 (▪) but not control antibody (Δ). (B) effect of elastase at various concentrations on OVCAR-3 cell survival. (C) SLPI reversal of elastase inhibition of OVCAR-3 cells (♦) is blocked by anti-SLPI antibody (◯). See Example 16 for more details.

FIG. 6: Effect of anti-SLPI antibody on the growth of SW480 colon carcinoma in athymic mice. Vehicle (□); CPT 100 mg/kg ip (Δ);anti-SLPI Mab 10 mg/kg iv (▾); anti-SLPI Mab 3 mg/kg iv (♦); anti-SLPI Mab 1 mg/kg iv (▴); and anti-SLPI Mab 3 mg/kg iv with CPT 100 mg/kg ip (⋄). See Example 17 for more details.

### DETAILED DESCRIPTION OF THE INVENTION

Definitions

The term “antibody,” as used in this disclosure, refers to an immunoglobulin or a fragment or a derivative thereof, and encompasses any polypeptide comprising an antigen-binding site, regardless whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)₂, Fv, scFv, Fd, dAb, and other antibody fragments that retain antigen-binding function, i.e., the ability to bind SLP1 specifically. Typically, such fragments would comprise an antigen-binding domain.

The terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.”

An antigen-binding domain typically comprises an antibody light chain variable region (VL) and an antibody heavy chain variable region (VH), however, it does not necessarily have to comprise both. For example, a so-called Fd antibody fragment consists only of a VH domain, but still retains some antigen-binding function of the intact antibody.

The term “repertoire” refers to a genetically diverse collection of nucleotides derived wholly or partially from sequences that encode expressed immunoglobulins. The sequences are generated by in vivo rearrangement of, e.g., V, D, and J segments for H chains and, e.g., V and J segment for L chains. Alternatively, the sequences may be generated from a cell line by in vitro stimulation, in response to which the rearrangement occurs. Alternatively, part or all of the sequences may be obtained by combining, e.g., unrearranged V segments with D and J segments, by nucleotide synthesis, randomised mutagenesis, and other methods, e.g., as disclosed in U.S. Pat. No.5,565,332.

The terms “specific interaction” and “specific binding” refer to two molecules forming a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the affinity constant K_{A} is higher than 10⁶ M⁻¹, or more preferably higher than 10⁸ M⁻¹. If necessary, non-specific binding can be reduced without substantially affecting specific binding by varying the binding conditions. The appropriate binding conditions such as concentration of antibodies, ionic strength of the solution, temperature, time allowed for binding, concentration of a blocking agent (e.g., serum albumin, milk casein), etc., may be optimized by a skilled artisan using routine techniques.

The phrase “substantially as set out” means that the relevant CDR, VH, or VL domain of the invention will be either identical to or have only insubstantial differences in the specified regions (e.g., a CDR), the sequence of which is set out. Insubstantial differences include minor amino acid changes, such as substitutions of 1 or 2 out of any 5 amino acids in the sequence of a specified region.

The term “SLPI activity” refers to one or more regulatory activities associated with SLPI. For example, SLPI inhibits the activity several proteases, including elastase and cathepsin G, trypsin and chymotrypsin. SLPI also stimulates the growth of human ovarian cancer cell lines. To “modulate” SLPI activity is to alter the baseline results observed with, and that can be attributed to SLPI. To “neutralize” SLPI is to cancel any effects, e.g. activity observed with, and that can be attributed to SLPI. Procedures for assessing the SLPI activity in vitro are described for example, in Examples 9, 10, 13, 15, 16, and 17.

The terms “treatment” and “therapeutic method” refer to both therapeutic treatment and prophylactic/preventative measures. Those in need of treatment may include individuals already having a particular medical disorder as well as those who may ultimately acquire the disorder (i.e., those needing preventative measures).

The term “effective amount” refers to a dosage or amount that is sufficient to reduce the activity of SLPI to result in amelioration of symptoms in a patient or to achieve a desired biological outcome.

The term “isolated” refers to a molecule that is substantially free of its natural environment. For instance, an isolated protein is substantially free of cellular material or other proteins from the cell or tissue source from which it is derived. The term “isolated” also refers to preparations where the isolated protein is sufficiently pure to be administered as a pharmaceutical composition, or at least 70-80% (w/w) pure, more preferably, at least 80-90% (w/w) pure, even more preferably, 90-95% pure; and, most preferably, at least 95%, 96%, 97%, 98%, 99%, or 100% (w/w) pure.

Unless otherwise defined, scientific and technical terms used in connection with the invention described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. (See e.g., Sambrook et al. Molecular Cloning: A Laboratory Manual, 2d ed., Cold Spring Harbor Laboratory Press, Cold Spring Harbor, N.Y. 1989). The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

Antibodies, also known as immunoglobulings, are typically tetrameric glycosylated proteins composed of two light (L) chains (about 25 kDa) each and two heavy (H) chains (about 50-70 kDa). The amino-terminal portion of each chain includes a variable domain of about 100 to 110 or more amino acids primarily responsible for antigen recognition. The carboxy-terminal portion of the L and H chain has one and three or four constant domains, respectively that are primarily responsible for effector function. There are two types of human L chains, classified as kappa and lambda. H chains are classified as mu, delta, gamma, alpha, or epsilon based upon the constant domain amino acid sequence, defining the antibody's isotype as IgM, IgD, IgG, IgA, and IgE, respectively. Isotypes may be further divided into subclasses e.g. IgG₁, IgG₂, IgG₃, IgG₄.

Immunoglobulins are produced in nature, in vivo by B lymphocytes. Each clone of B cells produces antibody with an antigen receptor having a unique prospective antigen binding structure. The repertoire of antigen receptors, approximately 10⁷ possibilities, exists in vivo prior to antigen stimulation. This diversity is produced by somatic recombination-the joining of different antibody gene segments. Immunoglobulin H chain, kappa L chain and lambda L chain are encoded by three separate genetic loci and each loci has multiple copies of at least 3 types of gene segments encoding variable (V), constant (C) and joining (J) regions, the heavy chain gene also includes a diversity (D) region. The selection of specific V, C and J regions (and D for the heavy chain) from amongst the various gene segments available (45 heavy chain V; 35 kappa V; 23 heavy chain D; 6 heavy chain J; 5 kappa J) generates approximately 10¹¹ possible specificities of germline sequences exhibited in B cells. The joining of V, C and J regions can result in the loss or addition of residues at the junctions. The L and H chain V region of human antibodies consists of relatively conserved framework regions (FR) that form a scaffold for three hypervariable regions also known as complementary determining regions (CDR). From the amino terminus of either the heavy or light chain, the V domain is made up of FR and CDR regions in the following order: FR1-CDR1-FR2-CDR2-FR3-CDR3-FR4. The CDRs are generally responsible for antigen binding.

The current invention provides germline human antibody heavy chain V, D, J combinations and light chain V, J combinations including nucleotide and amino acid sequence of the VH and VL domain FR and CDR regions with specificity for SLPI.

Upon exposure to antigen, those B cells with antigen binding specificity based on germline sequences are activated, proliferate, and differentiate to produce immunoglobulins of different isotypes as well as undergo somatic mutation and/or affinity maturation to produce immunoglobulins of higher affinity for the antigen. The current invention provides the nucleotide and amino acid sequence of such affinity matured V domain FR and CDR regions having specificity to SLPI.

Fab type Antibody fragments containing the antigen binding portion of the antibody molecule may consist of the L chain covalently linked by a disulfide bond to a portion of the H chain which has the V domain and first constant domain. Single chain Fv antibody fragment (scFv) has the H variable domain is linked to the L variable domain by a polypeptide linker. The invention provides antibody fragments such as Fab and scFv molecules having sequences derived from germline or affinity matured V domains of antibodies binding specifically to SLPI.

In a particular embodiment, human anti-SLPI antibodies are 42C1, 35F1, 43H7 and 9G12 and have amino acid sequences and nucleic acid sequences encoding them identified in this application as shown in Table 1.

TABLE 1
Antibody Nucleotide (DNA) and Amino Acid (AA) Sequences

| Gene Segment | 43H7 | 42C1 | 9G12 | 35F1 |
|---|---|---|---|---|
| H variable DNA | SEQ ID NO: 1 | SEQ ID NO: 19 | SEQ ID NO: 37 | SEQ ID NO: 55 |
| H variable AA | SEQ ID NO: 2 | SEQ ID NO: 20 | SEQ ID NO: 38 | SEQ ID NO: 56 |
| L variable DNA | SEQ ID NO: 10 | SEQ ID NO: 28 | SEQ ID NO: 46 | SEQ ID NO: 64 |
| L variable AA | SEQ ID NO: 11 | SEQ ID NO: 29 | SEQ ID NO: 47 | SEQ ID NO: 65 |
| H FR1 | SEQ ID NO: 3 | SEQ ID NO: 21 | SEQ ID NO: 39 | SEQ ID NO: 57 |
| H CDR1 | SEQ ID NO: 4 | SEQ ID NO: 22 | SEQ ID NO: 40 | SEQ ID NO: 58 |
| H FR2 | SEQ ID NO: 5 | SEQ ID NO: 23 | SEQ ID NO: 41 | SEQ ID NO: 59 |
| H CDR2 | SEQ ID NO: 6 | SEQ ID NO: 24 | SEQ ID NO: 42 | SEQ ID NO: 60 |
| H FR3 | SEQ ID NO: 7 | SEQ ID NO: 25 | SEQ ID NO: 43 | SEQ ID NO: 61 |
| H CDR3 | SEQ ID NO: 8 | SEQ ID NO: 26 | SEQ ID NO: 44 | SEQ ID NO: 62 |
| H FR4 | SEQ ID NO: 9 | SEQ ID NO: 27 | SEQ ID NO: 45 | SEQ ID NO: 63 |
| L FR1 | SEQ ID NO: 12 | SEQ ID NO: 30 | SEQ ID NO: 48 | SEQ ID NO: 66 |
| L CDR1 | SEQ ID NO: 13 | SEQ ID NO: 31 | SEQ ID NO: 49 | SEQ ID NO: 67 |
| L FR2 | SEQ ID NO: 14 | SEQ ID NO: 32 | SEQ ID NO: 50 | SEQ ID NO: 68 |
| L CDR2 | SEQ ID NO: 15 | SEQ ID NO: 33 | SEQ ID NO: 51 | SEQ ID NO: 69 |
| L FR3 | SEQ ID NO: 16 | SEQ ID NO: 34 | SEQ ID NO: 52 | SEQ ID NO: 70 |
| L CDR3 | SEQ ID NO: 17 | SEQ ID NO: 35 | SEQ ID NO: 53 | SEQ ID NO: 71 |
| L FR4 | SEQ ID NO: 18 | SEQ ID NO: 36 | SEQ ID NO: 54 | SEQ ID NO: 72 |

SLPI binding human antibodies may include H or L constant domains including L kappa or lambda constant regions, or any isotype H constant domain. In one embodiment of the invention, a human antibody with binding specificity to SLPI contains germline sequences such as the heavy chain V region VH4-31 (SEQ ID NOs:81. 82) or VH3-33 (SEQ ID NOs:83, 84); the heavy chain D region D2-2 (SEQ ID NOs:85, 86, 87, 88), D3-10 (SEQ ID NOs:89, 90, 91, 92), D4-17 (SEQ ID NOs:93, 94, 95, 96) or D3-16 (SEQ ID NOs:97, 98, 99, 100); the heavy chain J region JH4b (SEQ ID NOs: 101, 102) or JH6b (SEQ ID NOs: 103, 104); the light chain V kappa regions L2VK3 (SEQ ID NOs: 105, 106) or A2VK2 (SEQ ID NOs: 107, 108); and the J region JK1 (SEQ ID NOs: 109, 110) (generally, see Kabat Sequences of Proteins of Immunological Interest, National Institutes of Health, Bethesda, Md. 1987 and 1991; also see Chothia & Lesk 1987 J. Mol. Biol. 196:901-917; Chothia et al. 1989 Nature 342:878-883). In a particular embodiment, a human antibody 42C1, with binding specificity to SLPI has the heavy chain V region VH4-3 1; the heavy chain D region D2-2; the heavy chain J region JH4b; the light chain V kappa regions L2VK3; and the J region JK1. In another embodiment, a human antibody 35F1 with binding specificity to SLPI contains the heavy chain V region VH4-31; the heavy chain D region D3-10; the heavy chain J region JH4b; the light chain V kappa regions L2VK3; and the J region JK1. In yet another embodiment, a human antibody 43H7, with binding specificity for SLPI contains the heavy chain V region VH3-33; the heavy chain D region D4-17; the heavy chain J region JH6b; the light chain V kappa regions A2VK2; and the J region JK1. Additionally, a human antibody 9G12 of the invention, with binding specificity for SLPI contains the heavy chain V region VH3-33; the heavy chain D region D3-16; the heavy chain J region JH6b; the light chain V kappa regions A2VK2; and the J region JK1.

In an embodiment of the invention, the isolated antibody has a heavy chain variable region polypeptide comprising an amino acid sequence selected from the group consisting of SEQ ID NOs: 2, 20, 38, 56, 73, 74, 82 and 84. Such amino acid sequences may be encoded by nucleotide sequences selected from the group consisting of SEQ ID NOs: 1, 19, 37, 55, 81 and 83. In another embodiment, the invention provides an isolated antibody that specifically binds to SLPI and has a light chain variable region polypeptide comprising an amino acid sequence selected from the group consisting of SEQ ID NOs: 11, 29, 47, 65, 75, 76, 106 and 108. Such amino acid sequences may be encoded by nucleotide sequences selected from the group consisting of SEQ ID NOs: 10, 28, 46, and 64, 105 and 107. In yet another embodiment, the invention provides an isolated antibody that specifically binds to SLPI and has a heavy chain polypeptide comprising an amino acid sequence selected from the group consisting of SEQ ID NOs: 2, 20, 38, 56, 73, 74, 82 and 84 and has a light chain polypeptide comprising an amino acid sequence selected from the group consisting of SEQ ID NOs: 11, 29, 47, 65, 75, 76. 106 and 108. In yet another embodiment of the invention, anti-SLPI antibodies comprise at least one CDR of any of the H or L CDR polypeptide sequences SEQ ID NOs: 4, 6, 8, 13, 15, 17, 22, 24, 26, 31, 33, 35, 40, 42, 44, 49, 51, 53, 58, 60, 62, 67, 69, 71 and 80.

In a particular embodiment, SLPI binding human antibodies of the invention originating from germline V heavy chain region VH4-31 have an amino acid sequence:

QVQLQESGPGLVKPSQTLSLTCTVSGX₁SIX₂SGX₃YYWS
WX₄RQX₅PGKGLEWIG
YIYYSGSTYYNPSLKS RVTISX₆DTSKNQFSLKLSSVTAADTAVX₇YCAR
EGX₈YYDX₉GGFDY WGQGTLVTVSS

| | | |
|---|---|---|
| Where: | | |
| | X1 is G or V; | (SEQ ID NO: 73) |
| | X2 is S or K; | |
| | X3 is G or D; | |
| | X4 is I or V; | |
| | X5 is H or L; | |
| | X6 is V or I; | |
| | X7 is Y or F; | |
| | X8 is E or S; and | |
| | X9 is E or S. | |

Furthermore, in particular embodiments H chain CDR sequences are the germline VH4-31 CDR amino acid sequences:

| | | |
|---|---|---|
| CDR1: | SGGYYWS | (SEQ ID NO: 77) |
| CDR2: | YIYYSGSTYYNPSLKS | (SEQ ID NO: 24) |
| CDR3: | EGEYYDEGGFDY. | (SEQ ID NO: 26) |

After affinity maturation, SLPI binding human antibodies from VH4-31 germline V regions have FR and CDR amino acid sequences: SEQ ID NOs: 21-26, 57-63 (see Table 1).

In another particular embodiment, SLPI binding human antibodies of the invention originating from germline V heavy chain region VH3-33 have an amino acid sequence:

| QVQLVESGGGVVQPGRSLRLSCAASGFTFSX₁YGMHWVRQAPGKGLEWVX₂ | | |
|---|---|---|
| X₃IWYX₄GX₅X₆X₇YYX₈DSVKGRFTX₉SRDNSKNTLYLQMNSLRAEDTAVYYCAR | | |
| GDDYGDYHPHYYYYGX₁₀DV WGQGTTVTVSS | | |
| | Where: | |
| | X1 is S, T or G; | (SEQ ID NO: 74) |
| | X2 is A or T; | |
| | X3 is V or F; | |
| | X4 is D or N; | |
| | X5 is S, R or N; | |
| | X6 is N or D; | |
| | X7 is K or N; | |
| | X8 is A or T; | |
| | X9 is I or V; and | |
| | X10 is L or M. | |

Furthermore, in a particular embodiment, H chain CDR sequences are the germline VH3-33 CDR amino acid sequences:

| | | |
|---|---|---|
| CDR1: | SYGMH | (SEQ ID NO: 78) |
| CDR2: | VIWYDGSNKYYADSVKG | (SEQ ID NO: 79) |
| CDR3: | GDDYGDYHPHYYYYGLDV | (SEQ ID NO: 8) |

After affinity maturation, SLPI binding human antibodies from VH3-33 germline V heavy chain regions have FR and CDR amino acid sequences: SEQ ID NOs: 3-9, 39-45 (see Table 1).

In yet another particular embodiment, SLPI binding human antibodies of the invention originating from germline V light chain region L2VK3 have an amino acid sequence:

EIVMTQSPX₁TLSVSPGERATLSCRAX₂QSVX₃X₄NLAWYQQKPGQAPRLLIYGASTRAT
GIPARFSGSGSGTEFTLTISSLQSEDFAVYYCQQYNNWPPWTFGQGTKVEIX₅

| | |
|---|---|
| Where: | |
| X1 is A or T; | (SEQ ID NO: 75) |
| X2 is S or G; | |
| X3 is S or G; | |
| X4 is S or N; | |
| X5 is K or N. | |

Furthermore, in a particular embodiment, L chain CDR sequences are the germline L2VK3 CDR amino acid sequences:

| | | |
|---|---|---|
| CDR1: | RASQSVSSNLA | (SEQ ID NO: 31) |
| CDR2: | GASTRAT | (SEQ ID NO: 33) |
| CDR3: | QQYNNWPPWT | (SEQ ID NO: 35) |

After in vivo affinity maturation, SLPI binding human antibodies from L2VK3 germline V regions have FR and CDR amino acid sequences: SEQ ID NOs:30-36, 66-72 (see Table 1).

In a particular embodiment, SLPI binding human antibodies of the invention originating from germline V light chain region A2VK2 have an amino acid sequence:

DIVMTQTPLSLSVTPGQPASISCKSSQSLLX₁SDGKTYLYWYLQKPGQPPQLLIY
EVSNRFSGVPDRFSGSGSGTDFTLKISRVEAEDX₂X₃X₄YYCX₅QSIQLPWTFGQGTKVEIK

| | |
|---|---|
| Where: | |
| X1 is H or D; | (SEQ ID NO: 76) |
| X2 is V or I; | |
| X3 is G or A; | |
| X4 is V or Y; and | |
| X5 is M or L; | |

Furthermore, in a particular embodiment, L chain CDR sequences are the germline A2VK2 amino acid sequences:

| | | |
|---|---|---|
| CDR1: | KSSQSLLHSDGKTYLY | (SEQ ID NO: 80) |
| CDR2: | EVSNRFS | (SEQ ID NO: 15) |
| CDR3: | MQSIQLPWT | (SEQ ID NO: 17) |

After affinity maturation, SLPI binding human antibodies from A2VK2 germline V light heavy chain regions have FR and CDR amino acid sequences: SEQ ID NOs: 12-18, 48-54 (see Table 1).

In certain preferred embodiments of the invention, anti-SLPI antibodies modulate the activity of SLPI as demonstrated herein. In even more preferred embodiments of the invention, anti-SLPI antibodies neutralize the activity of SLPI, for example SLPI inhibition of elastase or cathepsin G enzyme activity, as demonstrated herein.

The antibodies of the invention bind an epitope of SLPI (SEQ ID NO: 112), preferably within the mature sequence of SLPI (aa 25-131 of SEQ ID NO:112). Antibodies of the invention bind SLPI with an affinity of 10⁻⁶ to 10⁻¹¹. Preferably with an affinity of 10⁻⁷ or greater and even more preferably 10⁻⁸ or greater. In a preferred embodiment, antibodies described herein bind to SLPI with very high affinities (Kd), for example a human antibody that is capable of binding SLPI with a Kd less than, but not limited to, 10⁻⁷, 10⁻⁸, 10⁻⁹, 10⁻¹⁰, 10⁻¹¹, 10⁻¹², 10¹³ or 10⁻¹⁴ M, or any range or value therein. Affinity and/or avidity measurements can be measured by KinExA® and/or BIACORE®, as described herein. In particular embodiments antibodies of the invention bind to SLPI with Kds ranging from 50 to 150 pM.

Epitope mapping and secondary and tertiary structure analyses can be carried out to identify specific 3D structures assumed by the disclosed antibodies and their complexes with antigens (see, e.g., Epitope Mapping Protocols, ed. Morris, Humana Press, 1996). Such methods include, but are not limited to, X-ray crystallography (Biochem. Exp. Biol., 11:7-13, 1974) and computer modeling of virtual representations of the presently disclosed antibodies (Fletterick et al. (1986) Computer Graphics and Molecular Modeling, in Current Communications in Molecular Biology, Cold Spring Harbor Laboratory, Cold Spring Harbor, N.Y.).

Furthermore, the specific part of the protein immunogen recognized by antibody may be determined by assaying the antibody reactivity to parts of the protein, for example an N terminal and C terminal half. The resulting reactive fragment can then be further dissected, assaying consecutively smaller parts of the immunogen with the antibody until the minimal reactive peptide is defined. Alternatively, the binding specificity, that is the epitope, of anti-SLPI antibodies of the invention may be determined by subjecting SLPI immunogen to SDS-PAGE either in the absence or presence of a reduction agent and analyzed by immunoblotting. Epitope mapping may also be performed using SELDI. SELDI ProteinChip® (LumiCyte) arrays used to define sites of protein-protein interaction. SLPI protein antigen or fragments thereof may be specifically captured by antibodies covalently immobilized onto the PROTEINCHIP array surface. The bound antigens may be detected by a laser-induced desorption process and analyzed directly to determine their mass.

The epitope recognized by anti-SLPI antibodies described herein may be determined by exposing the PROTEINCHIP Array to a combinatorial library of random peptide 12-mer displayed on Filamentous phage (New England Biolabs). Antibody-bound phage are eluted and then amplified and taken through additional binding and amplification cycles to enrich the pool in favor of binding sequences. After three or four rounds, individual binding clones are further tested for binding by phage ELISA assays performed on antibody-coated wells and characterized by specific DNA sequencing of positive clones.

Derivatives

This disclosure also provides a method for obtaining an antibody specific for SLPI. CDRs in such antibodies are not limited to the specific sequences of H and L variable domains identified in Table 1 and may include variants of these sequences that retain the ability to specifically bind SLPI. Such variants may be derived from the sequences listed in Table 1 by a skilled artisan using techniques well known in the art. For example, amino acid substitutions, deletions, or additions, can be made in the FRs and/or in the CDRs. While changes in the FRs are usually designed to improve stability and immunogenicity of the antibody, changes in the CDRs are typically designed to increase affinity of the antibody for its target. Variants of FRs also include naturally occurring immunoglobulin allotypes. Such affinity-increasing changes may be determined empirically by routine techniques that involve altering the CDR and testing the affinity of the antibody for its target. For example, conservative amino acid substitutions can be made within any one of the disclosed CDRs. Various alterations can be made according to the methods described in the art (Antibody Engineering, 2. sup. nd ed., Oxford University Press, ed. Borrebaeck, 1995). These include but are not limited to nucleotide sequences that are altered by the substitution of different codons that encode a functionally equivalent amino acid residue within the sequence, thus producing a “silent” change. For example, the nonpolar amino acids include alanine, leucine, isoleucine, valine, proline, phenylalanine, tryptophan, and methionine. The polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Substitutes for an amino acid within the sequence may be selected from other members of the class to which the amino acid belongs (see Table 2). Furthermore, any native residue in the polypeptide may also be substituted with alanine (Acta Physiol. Scand. Suppl. 643:55-67, 1998; Adv. Biophys. 35:1-24, 1998).

TABLE 2

Amino acid substitutions

| Original aa | | Prefered |
|---|---|---|
| Residue | Possible Substitutions | substitution |
| Ala (A) | Val, Leu, Ile | Val |
| Arg (R) | Lys, Gln, Asn | Lys |
| Asn (N) | Gln | Gln |
| Asp (D) | Glu | Glu |
| Cys (C) | Ser, Ala | Ser |
| Gln (Q) | Asn | Asn |
| Gly (G) | Pro, Ala | Ala |
| His (H) | Asn, Gln, Lys, Arg | Arg |
| Ile (I) | Leu, Val, Met, Ala, Phe, | Leu |
| | Norleucine | |
| Leu (L) | Norleucine, Ile, Val, Met, | Ile |
| | Ala, Phe | |
| Lys (K) | Arg, 1,4-Diamino-butyric | Arg |
| | Acid, Gln, Asn | |
| Met (M) | Leu, Phe, Ile | Leu |
| Phe (F) | Leu, Val, Ile, Ala, Tyr | Leu |
| Pro (P) | Ala Gly | Gly |
| Ser (S) | Thr, Ala, Cys | Thr |
| Thr (T) | Ser | Ser |
| Trp (W) | Tyr, Phe | Tyr |
| Tyr (Y) | Trp, Phe, Thr, Ser | Phe |
| Val (V) | Ile, Met, Leu, Phe, | Leu |
| | Ala, Norleucine | |

Derivatives and analogs of antibodies of the invention can be produced by various techniques well known in the art, including recombinant and synthetic methods (Maniatis (1990) Molecular Cloning, A Laboratory Manual, 2.sup.nd ed., Cold Spring Harbor Laboratory, Cold Spring Harbor, N.Y, and Bodansky et al. (1995) The Practice of Peptide Synthesis, 2.sup.nd ed., Spring Verlag, Berlin, Germany).

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts). A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in the art (for example, Proteins, Structures and Molecular Principles (Creighton, Ed., W. H. Freeman and Company, New York (1984)).

In one embodiment, a method for making an H variable domain which is an amino acid sequence variant of an H variable domain of the invention comprises a step of adding, deleting, substituting, or inserting one or more amino acids in the amino acid sequence of the presently disclosed H variable domain, optionally combining the H variable domain thus provided with one or more L variable domains, and testing the H variable domain or H variable/L variable combination or combinations for specific binding to SLPI or and, optionally, testing the ability of such antigen-binding domain to modulate SLPI activity. The L variable domain may have an amino acid sequence that is identical or is substantially as set out according to Table 1.

An analogous method can be employed in which one or more sequence variants of a L variable domain disclosed herein are combined with one or more H variable domains.

A further aspect of the disclosure provides a method of preparing antigen-binding fragment that specifically binds with SLPI. The method comprises:
(a) providing a starting repertoire of nucleic acids encoding a H variable domain that either includes a CDR3 to be replaced or lacks a CDR3 encoding region;
(b) combining the repertoire with a donor nucleic acid encoding an amino acid sequence substantially as set out herein for a H variable CDR3 such that the donor nucleic acid is inserted into the CDR3 region in the repertoire, so as to provide a product repertoire of nucleic acids encoding a H variable domain;
(c) expressing the nucleic acids of the product repertoire;
(d) selecting a binding fragment specific for SLPI; and
(e) recovering the specific binding fragment or nucleic acid encoding it.

Again, an analogous method may be employed in which a L variable CDR3 of the invention is combined with a repertoire of nucleic acids encoding a L variable domain, which either include a CDR3 to be replaced or lack a CDR3 encoding region. The donor nucleic acid may be selected from nucleic acids encoding an amino acid sequence substantially as set out in SEQ ID NOs:4, 6, 8, 13, 15, 17, 22, 24, 26, 31, 33, 35, 40, 42, 44, 49, 51, 53, 58, 60, 62, 67, 69, 71, 77, 78, 79 or 80.

A sequence encoding a CDR of the invention (e.g., CDR3) may be introduced into a repertoire of variable domains lacking the respective CDR (e.g., CDR3), using recombinant DNA technology, for example, using methodology described by Marks et al. (Bio/Technology (1992) 10: 779-783). In particular, consensus primers directed at or adjacent to the 5′ end of the variable domain area can be used in conjunction with consensus primers to the third framework region of human H variable genes to provide a repertoire of H variable domains lacking a CDR3. The repertoire may be combined with a CDR3 of a particular antibody. Using analogous techniques, the CDR3-derived sequences may be shuffled with repertoires of H variable or L variable domains lacking a CDR3, and the shuffled complete H variable or L variable domains combined with a cognate L variable or H variable domain to make the SLPI specific antibodies of the invention. The repertoire may then be displayed in a suitable host system such as the phage display system such as described in WO92/01047 so that suitable antigen-binding fragments can be selected.

Analogous shuffling or combinatorial techniques may be used (e.g. Stemmer, Nature (1994) 370: 389-391). In further embodiments, one may generate novel H variable or L variable regions carrying one or more sequences derived from the sequences disclosed herein using random mutagenesis of one or more selected H variable and/or L variable genes, such as error-prone PCR (Proc. Nat. Acad. Sci. U.S.A. (1992) 89: 3576-3580). Another method that may be used is to direct mutagenesis to CDRs of H variable or L variable genes (Proc. Nat. Acad. Sci. U.S.A. (1994) 91: 3809-3813; J. Mol. Biol. (1996) 263: 551-567). Similarly, one or more, or all three CDRs may be grafted into a repertoire of H variable or L variable domains, which are then screened for an antigen-binding fragment specific for SLPI.

A portion of an immunoglobulin variable domain will comprise at least one of the CDRs substantially as set out herein and, optionally, intervening framework regions as set out herein. The portion may include at least about 50% of either or both of FR1 and FR4, the 50% being the C-terminal 50% of FR1 and the N-terminal 50% of FR4. Additional residues at the N-terminal or C-terminal end of the substantial part of the variable domain may be those not normally associated with naturally occurring variable domain regions. For example, construction of antibodies by recombinant DNA techniques may result in the introduction of N- or C-terminal residues encoded by linkers introduced to facilitate cloning or other manipulation steps. Other manipulation steps include the introduction of linkers to join variable domains to further protein sequences including immunoglobulin heavy chain constant regions, other variable domains (for example, in the production of diabodies), or proteinaceous labels as discussed in further detail below.

Although the embodiments illustrated in the Examples comprise a “matching” pair of H variable and L variable domains, a skilled artisan will recognize that alternative embodiments may comprise antigen-binding fragments containing only a single CDR from either L variable or H variable domain. Either one of the single chain specific binding domains can be used to screen for complementary domains capable of forming a two-domain specific antigen-binding fragment capable of, for example, binding to SLPI. The screening may be accomplished by phage display screening methods using the so-called hierarchical dual combinatorial approach disclosed in WO92/01047, in which an individual colony containing either an H or L chain clone is used to infect a complete library of clones encoding the other chain (L or H) and the resulting two-chain specific binding domain is selected in accordance with phage display techniques as described.

Anti-SLPI antibodies described herein can be linked to another functional molecule, e.g., another peptide or protein (albumin, another antibody, etc.), toxin, radioisotope, cytotoxic or cytostatic agents. For example, the antibodies can be linked by chemical cross-linking or by recombinant methods. The antibodies may also be linked to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192; or 4,179,337. The antibodies can be chemically modified by covalent conjugation to a polymer, for example, to increase their circulating half-life. Exemplary polymers and methods to attach them are also shown in U.S. Pat. Nos. 4,766,106; 4,179,337; 4,495,285, and 4,609,546.

The disclosed antibodies may also be altered to have a glycosylation pattern that differs from the native pattern. For example, one or more carbohydrate moieties can be deleted and/or one or more glycosylation sites added to the original antibody. Addition of glycosylation sites to the presently disclosed antibodies may be accomplished by altering the amino acid sequence to contain glycosylation site consensus sequences known in the art. Another means of increasing the number of carbohydrate moieties on the antibodies is by chemical or enzymatic coupling of glycosides to the amino acid residues of the antibody (WO 87/05330; CRC Crit. Rev. Biochem., 22: 259-306, 1981). Removal of any carbohydrate moieties from the antibodies may be accomplished chemically or enzymatically (Arch. Biochem. Biophys., 259: 52,1987; Anal. Biochem., 118: 131, 1981; Meth. Enzymol., 138: 350, 1987). The antibodies may also be tagged with a detectable, or functional, label. Detectable labels include radiolabels such as ¹³¹I or ⁹⁹Tc, which may also be attached to antibodies using conventional chemistry. Detectable labels also include enzyme labels such as horseradish peroxidase or alkaline phosphatase. Detectable labels further include chemical moieties such as biotin, which may be detected via binding to a specific cognate detectable moiety, e.g., labeled avidin.

The valency of the antibodies may be custom designed to affect affinity and avidity, retention time at binding sites (see e.g. Am H. Pathol, 2002 160:1597-1608; J. Med. Chem. 2002 45:2250-2259; Br. J. Cancer 2002 86:1401-1410; Biomol. Eng. 2001 18:95-108; Int J. Cancer 2002 100:367-374).

Multiple specificity (bifunctional) binding reagents may be designed based upon the SLPI specific sequences of the invention (Biomol. Eng.2001 18:31-40). For example, a bispecific or bifunctional antibody is an artificial hybrid antibody having two different heavy/light chain pairs and two different binding sites. Bispecific antibodies can be produced by a variety of methods including fusion of hybridomas or linking of Fab′ fragments (Clin. Exp. Immunol. 1990, 79: 315-321;, J. Immunol. 199,2148:1547-1553).

Such bispecific antibodies can be generated comprising a specificity to SLPI and a second specificity to a second molecule using techniques that are well known (Immunol Methods 1994,4:72-81; Wright and Harris, supra.; Traunecker et al. 1992 Int. J. Cancer (Suppl.) 7:51-52). In one embodiment, the second specificity can be made to the heavy chain activation receptors, including, without limitation, CD16 or CD64 (Deo et al. 1997 18:127) or CD89 (Valerius et al. 1997 Blood 90:4485-4492). Bispecific antibodies prepared in this manner selectively kill cells expressing SLPI.

Antibodies, in which CDR sequences differ only insubstantially from those set out in SEQ ID NOs:4, 6, 8, 13, 15, 17, 22, 24, 26, 31, 33, 35, 40, 42, 44,49, 51, 53, 58, 60, 62, 67, 69, 71, 77, 78, 79 and 80, are encompassed within the scope of this invention. Typically, an amino acid is substituted by a related amino acid having similar charge, hydrophobic, or stereochemical characteristics. Such substitutions would be within the ordinary skills of an artisan. Unlike in CDRs, more substantial changes can be made in FRs without adversely affecting the binding properties of an antibody. Changes to FRs include, but are not limited to engineering certain framework residues that are important for antigen contact or for stabilizing the binding site, e.g., changing the class or subclass of the constant region, changing specific amino acid residues which might alter the effector function such as Fc receptor binding (U.S. Pat. Nos. 5,624,821; 5,648,260; Lund et al. (1991) J. Immun. 147: 2657-2662; Morgan et al. (1995) Immunology 86: 319-324), or changing the species from which the constant region is derived.

One of skill in the art will appreciate that the derivatives and modifications described above are not all-exhaustive, and that many other modifications would obvious to a skilled artisan in light of the teachings of the present disclosure.

Nucleic Acids, Cloning and Expression Systems

The present disclosure further provides isolated nucleic acids encoding the disclosed antibodies. The nucleic acids may comprise DNA or RNA and may be wholly or partially synthetic or recombinant. Reference to a nucleotide sequence as set out herein encompasses a DNA molecule with the specified sequence, and encompasses a RNA molecule with the specified sequence in which U is substituted for T, unless context requires otherwise.

The nucleic acids provided herein comprise a coding sequence for a CDR, a H variable domain, and/or a L variable domain disclosed herein.

The present disclosure also provides constructs in the form of plasmids, vectors, phagemids, transcription or expression cassettes which comprise at least one nucleic acid encoding a CDR, a H variable domain, and/or a L variable domain disclosed here.

The disclosure further provides a host cell which comprises one or more constructs as above.

Also provided are nucleic acids encoding any CDR (CDR1, CDR2, CDR3 from either the H or L variable domain), H variable or L variable domain, as well as methods of making of the encoded products. The method comprises expressing the encoded product from the encoding nucleic acid. Expression may be achieved by culturing under appropriate conditions recombinant host cells containing the nucleic acid. Following production by expression, a H variable or L variable domain, or specific binding member may be isolated and/or purified using any suitable technique, then used as appropriate.

Antigen-binding fragments, H variable and/or L variable domains and encoding nucleic acid molecules and vectors may be isolated and/or purified from their natural environment, in substantially pure or homogeneous form, or, in the case of nucleic acid, free or substantially free of nucleic acid or genes of origin other than the sequence encoding a polypeptide with the required function.

Systems for cloning and expression of a polypeptide in a variety of different host cells are well known in the art including cells suitable for producing antibodies (Gene Expression Systems, Academic Press, eds. Fernandez et al., 1999). Briefly, suitable host cells include bacteria, plant cells, mammalian cells, and yeast and baculovirus systems. Mammalian cell lines available in the art for expression of a heterologous polypeptide include Chinese hamster ovary cells, HeLa cells, baby hamster kidney cells, NSO mouse myeloma cells, and many others. A common bacterial host is E. coli. Any protein expression system compatible with the invention may be used to produce the disclosed antibodies. Suitable expression systems also include transgenic animals (Gene Expression Systems, Academic Press, eds. Fernandez et al., 1999).

Suitable vectors can be chosen or constructed, so that they contain appropriate regulatory sequences, including promoter sequences, terminator sequences, polyadenylation sequences, enhancer sequences, marker genes and other sequences as appropriate. Vectors may be plasmids or viral, e.g., phage, or phagemid, as appropriate (see Sambrook et al., Molecular Cloning: A Laboratory Manual, 2.sup.nd ed., Cold Spring Harbor Laboratory Press, 1989). Many known techniques and protocols for manipulation of nucleic acid, for example, in preparation of nucleic acid constructs, mutagenesis, sequencing, introduction of DNA into cells and gene expression, and analysis of proteins, are known in the art (Current Protocols in Molecular Biology, 2.sup.nd Edition, eds. Ausubel et al., John Wiley & Sons, 1992).

The invention also provides a host cell comprising a nucleic acid as disclosed herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage. The introduction of the nucleic acid into the cells may be followed by causing or allowing expression from the nucleic acid, e.g., by culturing host cells under conditions for expression of the gene.

Methods of Use

The disclosed anti-SLPI antibodies are capable of modulating and/or neutralizing the SLPI-associated inhibition of enzymes such as elastase and cathepsin G. The disclosed antibodies can act as either agonists or antagonists of SLPI, depending on the method of their use. The antibodies can be used to prevent, diagnose, or treat medical disorders in mammals, especially in humans. Antibodies of the invention can also be used for isolating SLPI or SLPI-expressing cells. Furthermore, the antibodies can be used to treat a subject at risk of or susceptible to a disorder or having a disorder associated with aberrant SLPI expression or function. Antibodies of the invention can be used to detect SLPI in such subjects.

The antibodies or antibody compositions of the present invention are administered in therapeutically effective amounts. Generally, a therapeutically effective amount may vary with the subject's age, condition, and sex, route of administration as well as the severity of the medical condition of the subject. A therapeutically effective amount of antibody ranges from about 0.001 to about 30 mg/kg body weight, preferably from about 0.01 to about 25 mg/kg body weight, from about 0.1 to about 20 mg/kg body weight, or from about 1 to about 10 mg/kg. The dosage may be adjusted, as necessary, to suit observed effects of the treatment. The appropriate dose is chosen based on clinical indications by a treating physician. The antibodies may given as a bolus dose, to maximize the circulating levels of antibodies for the greatest length of time after the dose. Continuous infusion may also be used.

In another aspect, the antibodies of the invention can be used as a targeting agent for delivery of another therapeutic or a cytotoxic agent (e.g., a toxin) to a cell expressing SLPI. The method includes administering an anti-SLPI antibody coupled to a therapeutic or a cytotoxic agent or under conditions that allow binding of the antibody to SLPI.

The antibodies of the invention may also be used to detect the presence of SLPI in biological samples. The amount of SLPI detected may be correlated with the expression level of SLPI, which, in turn, is correlated with the disease, tumor type, tumor burden or stage using methods known in the art (see for example recommendations of the AAPS Ligand Binding Assay Bioanalytical Focus Group (LBABFG) Pharm Res. November 2003;20(11):1885-900). Detection methods that employ antibodies are well known in the art and include, for example, ELISA, radioimmunoassay, immunoblot, Western blot, immunofluorescence, immunoprecipitation. The antibodies may be provided in a diagnostic kit that incorporates one or more of these techniques to detect SLPI. Such a kit may contain other components, packaging, instructions, or other material to aid the detection of the protein.

Where the antibodies are intended for diagnostic purposes, it may be desirable to modify them, for example, with a ligand group (such as biotin) or a detectable marker group (such as a fluorescent group, a radioisotope or an enzyme). If desired, the antibodies of the invention may be labeled using conventional techniques. Suitable detectable labels include, for example, fluorophores, chromophores, radioactive atoms, electron-dense reagents, enzymes, and ligands having specific binding partners. Enzymes are typically detected by their activity. For example, horseradish peroxidase can be detected by its ability to convert tetramethylbenzidine (TMB) to a blue pigment, quantifiable with a spectrophotometer. For detection, suitable binding partners include, but are not limited to, biotin and avidin or streptavidin, IgG and protein A, and the numerous receptor-ligand couples known in the art. Other permutations and possibilities will be readily apparent to those of ordinary skill in the art, -and are considered as equivalents within the scope of the instant invention.

Antibodies of the invention can be used in screening methods to identify inhibitors of SLPI effective as therapeutics. In such a screening assay, a first binding mixture is formed by combining SLPI and an antibody of the invention; and the amount of binding in the first binding mixture (M₀) is measured. A second binding mixture is also formed by combining SLPI, the antibody, and the compound or agent to be screened, and the amount of binding in the second binding mixture (M₁) is measured. A compound to be tested may be another anti-SLPI antibody. The amounts of binding in the first and second binding mixtures are then compared, for example, by calculating the M₁/M₀ ratio. The compound or agent is considered to be capable of modulating a SLPI-associated responses if a decrease in binding in the second binding mixture as compared to the first binding mixture is observed. The formulation and optimization of binding mixtures is within the level of skill in the art, such binding mixtures may also contain buffers and salts necessary to enhance or to optimize binding, and additional control assays may be included in the screening assay of the invention. Compounds found to reduce the SLPI-antibody binding by at least about 10% (i.e., M₁/M_{0<0.9}), preferably greater than about 30% may thus be identified and then, if desired, secondarily screened for the capacity to ameliorate a disorder in other assays or animal models as described below. The strength of the binding between SLPI and an antibody can be measured using, for example, an enzyme-linked immunoadsorption assay (ELISA), radio-immunoassay (RIA), surface plasmon resonance-based technology (e.g., Biacore), all of which are techniques well known in the art.

The compound may then be tested in vitro as described in the Examples Preliminary doses as, for example, determined according to animal tests, and the scaling of dosages for human administration is performed according to art-accepted practices. Toxicity and therapeutic efficacy can be determined by standard pharmaceutical procedures in cell cultures or experimental animals. The data obtained from the cell culture assays or animal studies can be used in formulating a range of dosage for use in humans. Therapeutically effective dosages achieved in one animal model can be converted for use in another animal, including humans, using conversion factors known in the art (see, e.g., Freireich et al. (1966) Cancer Chemother. Reports, 50(4): 219-244).

Pharmaceutical Compositions and Methods of Administration

The disclosure provides compositions comprising anti-SLPI antibodies. Such compositions may be suitable for pharmaceutical use and administration to patients. The compositions typically comprise one or more antibodies of the present invention and a pharmaceutically acceptable excipient. The phrase “pharmaceutically acceptable excipient” includes any and all solvents, dispersion media, coatings, antibacterial agents and antifungal agents, isotonic agents, and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration.

A pharmaceutical composition of the invention is formulated to be compatible with its intended route of administration. Methods to accomplish the administration are known to those of ordinary skill in the art. The administration may, for example, be intravenous, intraperitoneal, intramuscular, intracavity, subcutaneous or transdermal. It may also be possible to obtain compositions which may be topically or orally administered, or which may be capable of transmission across mucous membranes.

Solutions or suspensions used for intradermal or subcutaneous application typically include one or more of the following components: a sterile diluent such as water for injection, saline solution, fixed oils, polyethylene glycols, glycerin, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. The pH can be adjusted with acids or bases, such as hydrochloric acid or sodium hydroxide. Such preparations may be enclosed in ampoules, disposable syringes or multiple dose vials made of glass or plastic.

Pharmaceutical compositions suitable for injection include sterile aqueous solutions or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars; polyalcohols such as mannitol, sorbitol, and sodium chloride in the composition. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and/or by the use of surfactants. Prolonged absorption of the injectable compositions can be brought about by including in the composition an agent which delays absorption, for example, aluminum monostearate, and gelatin.

Oral compositions generally include an inert diluent or an edible carrier. They can be enclosed in gelatin capsules or compressed into tablets. For oral administration, the antibodies can be combined with excipients and used in the form of tablets, troches, or capsules. Pharmaceutically compatible binding agents, and/or adjuvant materials can be included as part of the composition. The tablets, pills, capsules, troches, and the like can contain any of the following ingredients, or compounds of a similar nature; a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, detergents, bile salts, and fusidic acid derivatives. Transmucosal administration may be accomplished, for example, through the use of lozenges, nasal sprays, inhalers, or suppositories. For example, in case of antibodies that comprise the Fc portion, compositions may be capable of transmission across mucous membranes in intestine, mouth, or lungs (e.g., via the FcRn receptor-mediated pathway as described in U.S. Pat. No. 6,030,613). For transdermal administration, the active compounds may be formulated into ointments, salves, gels, or creams as generally known in the art. For administration by inhalation, the antibodies may be delivered in the form of an aerosol spray from pressured container or dispenser, which contains a suitable propellant, e.g., a gas such as carbon dioxide, or a nebulizer.

In certain embodiments, the presently disclosed antibodies are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions containing the presently disclosed antibodies can also be used as pharmaceutically acceptable carriers. These can be prepared according to methods known to those skilled in the art, for example, as described in U.S. Pat. No. 4,522,811.

It may be advantageous to formulate oral or parenteral compositions in a dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of the composition of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

For any composition used in the present invention, the therapeutically effective dose can be estimated initially from cell culture assays. Examples of suitable bioassays include DNA replication assays, clonogenic assays and other assays as, for example, described in the Examples. The data obtained from the cell culture assays and animal studies can be used in formulating a range of dosage for use in humans. A dose may be formulated in animal models to achieve a circulating plasma concentration range that includes the IC₅₀ (i.e., the concentration of the antibody which achieves a half-maximal inhibition of symptoms). Circulating levels in plasma may be measured, for example, by high performance liquid chromatography. The effects of any particular dosage can be monitored by a suitable bioassay. The dosage lies preferably within a range of circulating concentrations with little or no toxicity. The dosage may vary depending upon the dosage form employed and the route of administration utilized.

Antibodies can be modified to become immunotoxins utilizing techniques that are well known in the art (Vitetta 1993, Immunol Today 14:252; U.S. Pat. No. 5,194,594). Cyotoxic immunoconjugates are known in the art and have been used as therapeutic agents. Such immunoconjugates may for example, use maytansinoids (U.S. Pat. No. 6,441,163), tubulin polymerization inhibitor, auristatin (Mohammad et al, 1999 Int. J. Oncol 15(2):367-72; Doronina et al, 2003 Nature Biotechnology 21(7):778-784), dolastatin derivatives (Ogawa et al, 2001 Toxicol Lett. 121(2):97-106) 21(3)778-784), Mylotarg®(Wyeth Laboratories, Philidelphia, Pa.); maytansinoids (DM1), taxane or mertansine (ImmunoGen Inc.).

Immunoradiopharmaceuticals utilizing anti-SLPI antibodies may be prepared utilizing techniques that are well known in the art (Junghans et al. in Cancer Chemotherapy and Biotherapy 655-686 (2d edition, Chafner and Longo, eds., Lippincott Raven (1996);U.S. Pat. Nos. 4,681,581, 4,735,210, 5,101,827, 5,102,990 (RE 35,500), U.S. Pat. No. 5,648,471, and 5,697,902). Each of the immunotoxins and radiolabeled antibody molecules selectively kill cells expressing SLPI. Radiolabels are known in the art and have been used for diagnostic or therapeutic radioimmuno conjugates. Examples of radiolabels includes, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I, ¹⁷⁷Lu, Rhenium-186, Rhenium-188 Samarium-153, Copper-64, Scandium-47). For example, radionuclides which have been used in radioimmunoconjugate guided clinical diagnosis include, but are not limited to: ¹³¹I, ¹²⁵I, ¹²³I, ⁹⁹Tc, ⁶⁷Ga, as well as ¹¹¹In. Antibodies have also been labeled with a variety of radionuclides for potential use in targeted immunotherapy (see Peirersz et al., 1987). These radionuclides include, for example, ¹⁸⁸Re and ¹⁸⁶ Re as well as ⁹⁰Y, and to a lesser extent ⁹⁹Au and ⁶⁷CU. I-(131) (see for example U.S. Pat. No. 5,460,785). Radiotherapeutic chelators and chelator conjugates are known in the art (U.S. Pat. No. 4,831,175, 5,099,069, 5,246,692, 5,286,850, and 5,124,471).

### EXAMPLES

The following examples, including the experiments conducted and results achieved are provided for illustrative purposes only and are not to be construed as limiting upon the present invention.

### Example 1

### Generation of Human Antibodies

A preferred method for generating fully human antibodies uses XenoMouse® strains of mice which have been engineered to contain 245 kb and 190 kb-sized germline configuration fragments of the human heavy chain locus and kappa light chain locus (Green et al. 1994 Nature Genetics 7:13-21; Mendez et al. 1997 Nature Genetics 15:146-156; Green and Jakobovits, 1998 J. Exp. Med. 188:483-495; U.S. Pat. Nos. 6,162,963, 6,150,584, 6,114,598, 6,075,181, and 5,939,598.) In an alternative approach, the minilocus approach, an exogenous Ig locus is mimicked through the inclusion of pieces (individual genes) from the Ig locus. Thus, one or more V_{H} genes, one or more DH genes, one or more J_{H} genes, a mu constant region, and a second constant region (preferably a gamma constant region) are formed into a construct for insertion into an animal (Taylor et al., 1992, Chen et al., 1993, Tuaillon et al., 1993, Choi et al., 1993, Lonberg et al., (1994), Taylor et al., (1994), and Tuaillon et al., (1995), Fishwild et al., (1996); U.S. Pat. Nos. 5,545,807, 5,545,806, 5,625,825, 5,625,126, 5,633,425, 5,661,016, 5,770,429, 5,789,650, 5,814,318, 5,877,397, 5,874,299, 6,255,458, 5,591,669, 6,023,010, 5,612,205, 5,721,367, 5,789,215, 5,643,763; 5,981,175). It is understood that the λκ XenoMouse® may be used to generate anti-SLPI antibodies utilizing lambda V regions. Such antibodies are within the scope of the invention.

### Example 2

### Immunization and Selection of Animals

Monoclonal antibodies specific for SLPI were developed by sequentially immunizing XenoMouse® mice (XenoMouse® strains XMG2, Abgenix, Inc. Fremont, Calif.) according to the schedule shown in Table 3 with recombinant human SLPI (R&D Systems, Minneapolis, Minn., Cat#260-PI). For instance, the initial immunization was with 10 μg antigen admixed 1:1 v/v with TiterMax® Gold. Subsequent boosts were made with 10 μg antigen admixed 1:1 v/v with 100 μg alum gel in pyrogen-free D-PBS and sometimes with 50% TiterMax® Gold, and then a final boost of 10 μg antigen in PBS. In particular, each mouse was immunized in the footpad by subcutaneous injection. The animals were immunized on days 0, 3, 8, 13, 17, 18, 21, 24, and 27. The animals were bled on days 16 and 23 to obtain sera for determining anti-SLPI titers.

TABLE 3
Immunization schedule for generating antibodies to SLPI
Antigen: Recombinant Human SLPI, Cat#260-PI (R&D Systems)

| | | | | | 1st | | | | | | | | | |
|---|---|---|---|---|---|---|---|---|---|---|---|---|---|---|
| Tar- | Mode | | # | Anti- | injec- | 2nd | 3rd | 4th | | 5th | 6th | | 7th | 8th |
| get | of Im. | Strain | mice | gen | tion | boost | boost | boost | Bleed | boost | boost | Bleed | boost | boost |
| SLPI | Foot- | XMG2 | 10 | SLPI | 10 μg/ | 10 μg/ | 10 μg/ | 10 μg/ | | 10 μg/ | 10 μg/ | | 10 μg/ | 10 μg/ |
| | pad | | | | mouse | mouse | mouse | mouse | | mouse | mouse | | mouse | mouse |
| | | | | | TiterMax | Alum | Alum | Alum | | Alum | TiterMax | | Alum | D-PBS |
| | | | | | Gold | Gel | Gel | Gel | | Gel | Gold | | Gel | |
| | | | | | Day 0 | Day 3 | Day 8 | Day 13 | Day 16 | Day 17 | Day 20 | Day 23 | Day 24 | Day 27 |

Anti-SLPI antibody titers were determined by indirect ELISA. Briefly, biotinylated SLPI (0.5 μg/mL) was coated onto Sigma Streptavidin Clear Polystyrene 96 well ELISA plates for 30 minutes at room temperature (RT). The plates were then washed 4× by hand, then pat dried on paper towels. XenoMouse® animal sera from SLPI immunized, or naïve XenoMouse® animals, were titrated out to 6 wells, the last well left blank, in 1% no fat skim milk/PBS at 1:2 dilutions in duplicate from a 1:500 initial dilution. 50 μl was used for each well. The plates were incubated for 1 hour then washed four times. 50 μl of a goat anti-human IgG Fc-specific HRP-conjugated antibody (0.4 μg/mL) was added to each well and incubated for 1 hour at RT. The plates were washed five times with distilled water (dH₂O). The plates were developed with the addition of TMB for 30 minutes and the ELISA was stopped by the addition of 1 M phosphoric acid. The specific titers of individual XenoMouse® animals were determined from the optical density at 450 nm, Table 2. The titers represent the reciprocal dilution of the serum to either SLPI or streptavidin and therefore the higher the number, the greater the humoral immune response. Lymph nodes were harvested for XenoMax® antibody generation from immunized XenoMouse® animals with specific titers to SLPI versus streptavidin as shown in Table 4.

TABLE 4
Titer of animals immunized with SLPI

| | Titer Biotinylated- | |
|---|---|---|
| Mouse I.Ds | SLPI @ 0.5 μg/ml | Titer SA |
| O154-1 | no data | no data |
| O154-2 | <100 @ half max OD | <100 @ half max OD |
| O154-3 | <100 @ half max OD | <100 @ half max OD |
| O154-4* | 2470 @ half max OD | <100 @ half max OD |
| O154-5 | 1488 @ half max OD | 1177 @ half max OD |
| O154-6 | <100 @ half max OD | <100 @ half max OD |
| O154-7 | <100 @ half max OD | <100 @ half max OD |
| O154-8 | <100 @ half max OD | <100 @ half max OD |
| O154-9* | 2790 @ half max OD | <100 @ half max OD |
| O154-10 | <100 @ half max OD | <100 @ half max OD |
| naïve | 787 @ half max OD | 902 @ half max OD |

*animals with specific titers to SLPI versus streptavidin

### Example 3

### XenoMax® Antibody Generation

Culture and Selection of B Cells:

B cells from the harvested animals were cultured and those secreting SLPI-specific antibodies were isolated as described previously (Babcook et al., 1996 Proc. Natl. Acad. Sci. USA, 93:7843-7848). ELISA, performed as described above, was used to identify primary SLPI-specific wells from fifty plates cultured at 500 or 150 cells/well. 55 wells (OD≧0.3) showed ODs significantly above background (0.1), as shown in Table 5.

TABLE 5
Selection of B cells specific for SLPI

| Plate | Wells | Positives above cutoff OD of: | | | | | | | | | | | |
|---|---|---|---|---|---|---|---|---|---|---|---|---|---|
| Breakdown | Screened | 0.1 | 0.2 | 0.3 | 0.4 | 0.5 | 0.6 | 0.7 | 0.8 | 0.9 | 1.0 | 1.5 | 2.0 |
| 500 cells/well | 2400 | 2314 | 524 | 38 | 16 | 7 | 6 | 5 | 4 | 4 | 3 | 1 | 1 |
| (plates #1-25) | | | | | | | | | | | | | |
| 150 cells/well | 2400 | 2126 | 257 | 17 | 12 | 8 | 7 | 6 | 6 | 4 | 4 | 3 | 2 |
| (plates #26-50) | | | | | | | | | | | | | |

These data indicated a very low frequency of wells and indicated that the wells were monoclonal for antigen-specificity at all cell dilutions. As shown in Table 6, These 55 positive wells were rescreened for binding to SLPI and only 33 wells were found to contain antigen specific antibody. The binding of these 33 wells were then ranked by limiting antigen analysis.

TABLE 6
SLPI specific antibody producing clones

| Plate | Well | | 1* OD | 2* OD |
|---|---|---|---|---|
| Cur12.1 PL43 | H* | 7 | 3.370 | 2.283 |
| Cur12.1 PL42 | C* | 1 | 3.227 | 2.246 |
| Cur12.1 PL09 | G* | 12 | 2.004 | 1.971 |
| Cur12.1 PL09 | A* | 12 | 0.471 | 1.964 |
| Cur12.1 PL45 | C* | 6 | 1.729 | 1.957 |
| Cur12.1 PL13 | G* | 5 | 1.297 | 1.369 |
| Cur12.1 PL47 | B* | 12 | 1.263 | 1.344 |
| Cur12.1 PL03 | F* | 11 | 0.909 | 1.229 |
| Cur12.1 PL11 | D* | 6 | 1.337 | 1.178 |
| Cur12.1 PL41 | E* | 2 | 0.552 | 1.134 |
| Cur12.1 PL47 | B* | 1 | 0.375 | 0.993 |
| Cur12.1 PL28 | A* | 12 | 0.304 | 0.937 |
| Cur12.1 PL08 | D* | 7 | 0.738 | 0.857 |
| Cur12.1 PL28 | H* | 9 | 0.825 | 0.800 |
| Cur12.1 PL15 | D* | 11 | 0.595 | 0.599 |
| Cur12.1 PL34 | A* | 4 | 0.419 | 0.590 |
| Cur12.1 PL40 | E* | 4 | 0.488 | 0.550 |
| Cur12.1 PL09 | A* | 2 | 0.670 | 0.541 |
| Cur12.1 PL16 | B* | 1 | 0.429 | 0.523 |
| Cur12.1 PL32 | G* | 2 | 0.418 | 0.484 |
| Cur12.1 PL36 | D* | 2 | 0.693 | 0.483 |
| Cur12.1 PL23 | E* | 6 | 0.422 | 0.438 |
| Cur12.1 PL08 | C* | 1 | 0.409 | 0.428 |
| Cur12.1 PL08 | E* | 1 | 0.308 | 0.413 |
| Cur12.1 PL35 | F* | 1 | 0.425 | 0.410 |
| Cur12.1 PL15 | A* | 8 | 0.311 | 0.378 |
| Cur12.1 PL19 | F* | 9 | 0.369 | 0.371 |
| Cur12.1 PL08 | B* | 1 | 0.350 | 0.366 |
| Cur12.1 PL50 | B* | 2 | 0.318 | 0.366 |
| Cur12.1 PL08 | A* | 1 | 0.391 | 0.365 |
| Cur12.1 PL09 | A* | 10 | 0.479 | 0.336 |
| Cur12.1 PL10 | A* | 2 | 0.340 | 0.322 |
| Cur12.1 PL05 | D* | 5 | 0.356 | 0.312 |
| Cur12.1 PL10 | B | 5 | 0.343 | 0.289 |
| Cur12.1 PL22 | F | 12 | 0.429 | 0.277 |
| Cur12.1 PL09 | A | 5 | 0.310 | 0.263 |
| Cur12.1 PL19 | D | 7 | 0.440 | 0.263 |
| Cur12.1 PL19 | B | 1 | 0.301 | 0.250 |
| Cur12.1 PL12 | A | 11 | 0.317 | 0.249 |
| Cur12.1 PL03 | E | 8 | 0.392 | 0.242 |
| Cur12.1 PL19 | B | 2 | 0.313 | 0.232 |
| Cur12.1 PL13 | A | 12 | 0.349 | 0.230 |
| Cur12.1 PL09 | A | 11 | 0.345 | 0.195 |
| Cur12.1 PL24 | C | 3 | 0.307 | 0.182 |
| Cur12.1 PL24 | D | 3 | 0.318 | 0.182 |
| Cur12.1 PL24 | E | 2 | 0.301 | 0.175 |
| Cur12.1 PL18 | G | 2 | 0.342 | 0.171 |
| Cur12.1 PL12 | A | 12 | 0.348 | 0.169 |
| Cur12.1 PL28 | A | 10 | 0.364 | 0.169 |
| Cur12.1 PL28 | A | 11 | 0.337 | 0.148 |
| Cur12.1 PL39 | A | 1 | 0.842 | 0.116 |
| Cur12.1 PL06 | A | 2 | 0.421 | Empty |
| Cur12.1 PL06 | A | 3 | 0.333 | Empty |
| Cur12.1 PL06 | B | 5 | 0.456 | Empty |
| Cur12.1 PL06 | D | 12 | 0.322 | Empty |

### Example 4

### Limiting Antigen Analysis

The limiting antigen analysis is a method that affinity ranks the antigen-specific antibodies in B cell culture supernatants. In the presence of very low concentrations of antigen, only the highest affinity antibodies will be able to bind to antigen with any detectable level at equilibrium (U.S. patent Publication 20030186327, published Oct. 2, 2003.) Biotinylated SLPI was coated on streptavidin plates for 30 minutes at RT, titrated 1:2 from 800 ng/ml down to 50 ng/ml. Each plate was washed 5 times with dH₂O, before 46 μl/well of 1% milk in PBS with 0.05% sodium azide was added, followed by 4 μl/well of B cell supernatant. After 22 hours at RT on a shaker, the plates were again washed 5 times with dH₂O. Goat anti-Human (Fc)-HRP at 1 μg/ml, 50 μl/well was added. After 1 hour at RT, the plates were again washed 5 times with dH₂O and 50 μl/well of TMB substrate was added. The reaction was stopped by the addition of 50 μl of 1M phosphoric acid to each well and the plates were read at wavelength 450 nm. Results are shown in Table 7. 42C1, 43H7, 35F1 and 9G12 were chosen for further evaluation and cloning.

TABLE 7
Antibody affinity ranking by limiting antigen analysis
Limiting Antigen Data:

| | Conc. of | | | | | | | | | |
|---|---|---|---|---|---|---|---|---|---|---|
| | B-Cur12 | | | | | | | | | |
| ID | 800 ng/ml | Rank | 400 ng/ml | Rank | 200 ng/ml | Rank | 100 ng/ml | Rank | 50 ng/ml | Rank |
| 42C1 | 3.85 | 2 | 3.58 | 2 | 3.69 | 1 | 3.42 | 1 | 3.01 | 1 |
| 43H7 | 4.10 | 1 | 4.02 | 1 | 3.52 | 2 | 2.60 | 2 | 1.19 | 2 |
| 9G12 | 2.25 | 4 | 2.19 | 3 | 1.36 | 3 | 0.98 | 3 | 0.60 | 3 |
| 45C6 | 2.77 | 3 | 1.98 | 4 | 1.29 | 4 | 0.79 | 4 | 0.49 | 4 |
| 47B12 | 0.91 | 11 | 1.08 | 9 | 0.87 | 7 | 0.61 | 6 | 0.45 | 5 |
| 11D6 | 1.68 | 5 | 1.52 | 5 | 1.03 | 5 | 0.67 | 5 | 0.43 | 6 |
| 8D7 | 0.55 | 13 | 0.72 | 11 | 0.61 | 8 | 0.56 | 8 | 0.35 | 7 |
| 13G5 | 1.32 | 8 | 1.24 | 6 | 0.89 | 6 | 0.59 | 7 | 0.33 | 8 |
| 36D2 | 0.29 | 17 | 0.15 | 21 | 0.22 | 14 | 0.21 | 12 | 0.28 | 9 |
| 40E4 | 0.31 | 16 | 0.47 | 12 | 0.44 | 9 | 0.34 | 9 | 0.27 | 10 |
| 10A2 | 0.18 | 20 | 0.17 | 18 | 0.17 | 16 | 0.18 | 16 | 0.20 | 11 |
| 9A12 | 0.15 | 21 | 0.20 | 17 | 0.20 | 15 | 0.22 | 11 | 0.17 | 12 |
| 35F1 | 0.37 | 15 | 0.30 | 14 | 0.24 | 13 | 0.20 | 13 | 0.17 | 13 |
| 9A10 | 0.15 | 22 | 0.16 | 20 | 0.16 | 19 | 0.17 | 17 | 0.16 | 14 |
| 3F11 | 1.55 | 6 | 1.21 | 7 | 0.37 | 11 | 0.20 | 15 | 0.15 | 15 |
| 28H9 | 1.10 | 9 | 0.77 | 10 | 0.32 | 12 | 0.22 | 10 | 0.14 | 16 |
| 8C1 | 0.11 | 23 | 0.11 | 27 | 0.12 | 22 | 0.13 | 20 | 0.13 | 17 |
| 19F9 | 0.42 | 14 | 0.29 | 15 | 0.15 | 20 | 0.14 | 19 | 0.13 | 18 |
| 8A1 | 0.11 | 24 | 0.12 | 22 | 0.11 | 24 | 0.13 | 22 | 0.13 | 19 |
| 15A8 | 0.19 | 18 | 0.12 | 24 | 0.11 | 25 | 0.11 | 25 | 0.13 | 20 |
| 16B1 | 0.93 | 10 | 0.40 | 13 | 0.17 | 18 | 0.14 | 18 | 0.12 | 21 |
| 15D11 | 0.09 | 28 | 0.11 | 26 | 0.10 | 29 | 0.11 | 27 | 0.12 | 22 |
| 5D5 | 0.19 | 19 | 0.16 | 19 | 0.17 | 17 | 0.13 | 21 | 0.12 | 23 |
| 9A2 | 0.11 | 26 | 0.11 | 25 | 0.11 | 23 | 0.12 | 24 | 0.11 | 24 |
| 8B1 | 0.11 | 25 | 0.12 | 23 | 0.13 | 21 | 0.13 | 23 | 0.11 | 25 |
| 41E2 | 1.33 | 7 | 1.17 | 8 | 0.42 | 10 | 0.20 | 14 | 0.11 | 26 |
| 47B1 | 0.08 | 32 | 0.08 | 32 | 0.09 | 32 | 0.08 | 33 | 0.11 | 27 |
| 8E1 | 0.10 | 27 | 0.10 | 28 | 0.10 | 28 | 0.11 | 26 | 0.10 | 28 |
| 28A12 | 0.09 | 29 | 0.10 | 29 | 0.10 | 26 | 0.11 | 29 | 0.10 | 29 |
| 32G2 | 0.08 | 30 | 0.09 | 30 | 0.09 | 30 | 0.09 | 30 | 0.10 | 30 |
| 34A4 | 0.59 | 12 | 0.25 | 16 | 0.09 | 31 | 0.09 | 31 | 0.10 | 31 |
| 23E6 | 0.08 | 31 | 0.09 | 31 | 0.10 | 27 | 0.11 | 28 | 0.08 | 32 |
| 50B2 | 0.06 | 33 | 0.06 | 33 | 0.07 | 33 | 0.08 | 32 | 0.07 | 33 |

### Example 5

### Neutralization and Binding Inhibition Assay

Antibodies were screened for ability to inhibit SLPI activity. The assay involves assessing SLPI binding to chymotrypsin and resultant inhibition of chymotrypsin enzyme activity in the presence or absence of antibody. Biotinylated bovine chymotrypsin (5 μg/ml) was incubated with streptavidin coated plates for 30 minutes at RT. Each plate was washed 5 times with dH₂O prior to addition of an anti-SLPI antibody mixture (30 μl of SLPI reconstituted in 100 mM Tris, 10 mM CaCl₂ and 0.1% HSA pH 7.5 mixed with 20 μl of monoclonal antibody supernatant being tested; final concentration SLPI 60 ng/ml; incubated for 1 hour at RT) and plates were incubated for 1 hour. Plates were washed 5 times with dH₂O then incubated with 50 μl of a polyclonal Goat anti-SLPI (purchased from R&D) at 1 μg/ml/well for 1 hour. After washing, 50 μl of a rabbit anti-goat HRP-conjugated antibody/well was added and incubated for 1 hour. After washing, 50 μl of TMB substrate/well was added.. The reaction was stopped by adding of 50 μl of 1M phosphoric acid/well. Plates were read at wavelength 450 nm. Results are shown in Table 8.

TABLE 8
Antibody SLPI neutralization activity
Neutralization cutoff 80%

| ID | O.D. | % Max | Neutralizer | Rank |
|---|---|---|---|---|
| 42C1 | 0.762 | 103% | NO | |
| 43H7 | 0.666 | 90% | NO | |
| 9G12 | 0.452 | 61% | YES | 6 |
| 45C6 | 0.616 | 83% | No | |
| 47B12 | 0.5 | 68% | YES | |
| 11D6 | 0.459 | 62% | YES | 7 |
| 8D7 | 0.706 | 96% | NO | |
| 13G5 | 0.806 | 109% | NO | |
| 36D2 | 0.703 | 95% | NO | |
| 40E4 | 0.829 | 112% | NO | |
| 10A2 | 0.418 | 57% | YES | 3 |
| 9A12 | 0.43 | 58% | YES | 4 |
| 35F1 | 0.35 | 47% | YES | 2 |
| 9A10 | 0.499 | 68% | YES | |
| 3F11 | 0.461 | 62% | YES | 7 |
| 28H9 | 0.516 | 70% | YES | |
| 8C1 | 0.571 | 77% | YES | |
| 19F9 | 0.563 | 76% | YES | |
| 8A1 | 0.59 | 80% | YES | |
| 15A8 | 0.722 | 98% | NO | |
| 16B1 | 0.64 | 87% | NO | |
| 15D11 | 0.885 | 120% | NO | |
| 5D5 | 0.999 | 135% | NO | |
| 9A2 | 0.479 | 65% | YES | 8 |
| 8B1 | 0.596 | 81% | No | |
| 41E2 | 0.916 | 124% | NO | |
| 47B1 | 0.701 | 95% | NO | |
| 8E1 | 0.677 | 92% | NO | |
| 28A12 | 0.435 | 59% | YES | 5 |
| 32G2 | 0.603 | 82% | YES | |
| 34A4 | 0.327 | 44% | YES | 1 |
| 23E6 | 0.713 | 96% | NO | |
| 50B2 | 0.576 | 78% | YES | |
| Irrelevant well 1 | 0.678 | 92% | N/A | |
| Irrelevant well 2 | 0.771 | 104% | N/A | |
| no SLPI, no BCC | 0.114 | 15% | N/A | |
| no SLPI, BCC | 0.108 | 15% | N/A | |
| SLPI, no BCC | 0.739 | 100% | N/A | |

Neutralization was calculated by taking the OD of each well and comparing it to the maximum achievable OD as judged by control wells without added supernatant.

### Example 6

### Isolation of Abs

SLPI-specific Hemolytic Plaque Assay:

Biotinylation of Sheep red blood cells (SRBCs). SRBCs are stored in RPMI media as a 25% stock. A 250 μl SRBC packed-cell pellet was prepared from 1.0 ml of the stock by spinning down the cells and removing the supernatant. The cell pellet was re-suspended in 4.75 ml PBS at pH 8.6.. In a separate 50 ml tube, 2.5 mg of Sulfo-NHS biotin was completely dissolved in 45 ml of PBS at pH 8.6 and 5 ml of SRBCs were added and the tube rotated at RT for 1 hour. The SRBCs were centrifuged at 3000 g for 5 min, the supernatant drawn off and cells were washed 3 times with 25 mls PBS at pH 7.4, followed by adding 4.75 ml immune cell media (RPMI 1640 with 10% FCS) re-suspending the B-SRBC (5% B-SRBC stock). Stock was stored at 4° C.

Streptavidin (SA) coating of B-SRBCs. The B-SRBC cells from 1 ml of the 5% B-SRBC were pelleted, washed twice in 1.0 ml PBS at pH 7.4, pelleted again with a pulse spin at 8000 rpm (6800 rcf) in a microfuge and resuspended in 1.0 ml of PBS at pH 7.4 to give a final concentration of 5% (v/v). 10 μl of a 10 mg/ml streptavidin (CalBiochem, San Diego, Calif.) stock solution was added and the tube mixed and rotated at RT for 20 min. The washing steps were repeated and the SA-SRBCs were re-suspended in 1 ml PBS pH 7.4 (5% (v/v)).

SLPI coating of SA-SRBCs. The SA-SRBCs were coated with biotinylated-SLPI at 10 μg/ml, the mixed and rotated at RT for 20 min. The SRBCs were washed twice with 1.0 ml of PBS at pH 7.4 as above. The SLPI-coated SRBCs were re-suspended in RPMI (+10%FCS) to a final concentration of 5% (v/v).

Determination of the quality of SLPI-SRBCs by immunofluorescence (IF). 10 μl of 5% SA-SRBCs and 10 μl of 5% SLPI-coated SRBCs were each added to separate tubes containing 40 μl of PBS. A control goat anti-SLPI antibody was added to each sample of SRBCs at 40 μg/ml. The tubes were rotated at RT for 25 min, and the cells were then washed three times with 100 μl of PBS and re-suspended in 50 μl of PBS, incubated with 40 μg/mL Rb-anti goat IgG Fc antibody conjugated to Alexa488 (Molecular Probes, Eugene, Oreg.). The tubes were rotated at RT for 25 min, cells were washed with 100 μg PBS and re-suspended in 10 μl PBS. 10 μl of stained cells were spotted onto a clean glass microscope slide, covered with a glass cover slip, observed under fluorescent light, and scored on a scale of 0-4 for immunofluorescent intensity.

Preparation of plasma cells. Single microculture wells, previously identified as containing a B cell clone secreting the immunoglobulin of interest, were harvested and transferred to a fresh tube (final vol. approx 500-700 μl). Cells were centrifuged at 1500 rpm (240 rcf) for 2 minutes at room temperature, then the tube rotated 180 degrees and spun again for 2 minutes at 1500 rpm. The freeze media was drawn off and the immune cells resuspended in 100 μl RPMI (10% FCS), then centrifuged. This washing with RPMI (10% FCS) was repeated and the cells re-suspended in 60 μl RPMI (FCS) and stored on ice until ready to use.

Plaque assay. A 60 μl sample of cells was mixed with 60 μl each of SLPI-coated SRBC (5% v/v stock), 4× guinea pig complement (Sigma, Oakville, ON) stock prepared in RPMI (FCS), and 4× enhancing sera stock (1:900 in RPMI (FCS)). The mixture (3-5 μl) was spotted onto silicone edge prepared glass slides (SigmaCoat, Sigma, Oakville, ON), covered with undiluted paraffin oil and incubated at 37° C. for a minimum of 45 minutes.

Results. SLPI coating of SRBC was determined qualitatively by immunofluorescent microscopy and found to be very high (4/4) with control goat anti-SLPI polyclonal antibody to detect a coating compared to a secondary detection reagent alone (0/4). There was no signal detected using a control goat anti-SLPI antibody on red blood cells that were only coated with streptavidin (0/4). These red blood cells were then used to identify antigen-specific plasma cells from wells 42C1, 43H7, 9G12 and 35F1. After micromanipulation to rescue the antigen-specific plasma cells, the genes encoding the variable region genes were rescued by RT-PCR on a single plasma cell.

### Example 7

### Expression of SLPI Antibodies

After isolation of single plasma cells, mRNA was extracted and reverse transcriptase PCR was used to generate cDNA. The cDNA encoding the variable heavy and light chains was specifically amplified using polymerase chain reaction. The variable heavy chain region was cloned into an IgG2 expression vector, generated by cloning the constant domain of human IgG2 into the multiple cloning site of pcDNA3.1+/Hygro (Invitrogen, Burlington, ON). The variable light chain region was cloned into an IgK expression vector, generated by cloning the constant domain of human IgK into the multiple cloning site of pcDNA3.1+/Neo (Invitrogen, Burlington, ON). The heavy chain and the light chain expression vectors were then co-lipofected into a 60 mm dish of 70% confluent human embryonic kidney 293 (HEK 293) cells and the transfected cells were incubated for 24 hours to secrete recombinant antibody. Supernatant (3 mL) was harvested from the HEK 293 cells and the secretion of intact antibody and binding to SLPI tested ELISA. To detection secretion, 96 well plates were coated with 2 μg/mL of goat anti-human IgG H+L overnight. For detection of SLPI binders, streptavidin plates were coated with biotinylated human SLPI (0.5 μg/mL) overnight at four degrees. The plates were washed five times with dH₂O. Recombinant antibodies titrated 1:2 for 7 wells from the undiluted minilipofection supernatant were added, incubated and the plates were washed five times with dH₂O. Goat anti-human IgG Fc-specific HRP-conjugated antibody was added at a final concentration of 1 μg/mL, incubated for 1 hour at RT and the plates were washed as before. Plates were developed with the addition of TMB for 30 minutes and the reaction was stopped by the addition of 1 M phosphoric acid. Each ELISA plate was analyzed to determine the optical density of each well at 450 nm. Results are shown in Table 9.

TABLE 9
Recombinant anti-SLPI antibodies

| Clone | Human IgG | Binding to SLPI |
|---|---|---|
| 35F1 | >1:64 | >1:64 |
| 42C1 | 1:64 | >1:64 |
| 43H7 | 1:16 | >1:64 |
| 9G12 | 1:32 | >1:64 |

### Example 8

### Structural Analysis of Anti-SLPI Antibodies

The variable heavy chains and the variable light chains for the antibodies shown in Table 9 were sequenced to determine their DNA and protein sequences.

Antibody -43H7

Heavy chain variable region

Nucleotide sequence

| | |
|---|---|
| 5′CAGGTGCAGCTGGTGGAGTCTGGGGGAGGCGTGGTCCAGCCTGGGAGGTC | (SEQ ID NO: 1) |
| CCTGAGACTCTCCTGTGCAGCGTCTGGATTCACCTTCAGTACCTATGGCATGC | |
| ACTGGGTCCGCCAGGCTCCAGGCAAGGGGCTGGAGTGGGTGACATTTATATG | |
| GTATGATGGACGTAATAAATACTATGCAGACTCCGTGAAGGGCCGATTCACC | |
| GTCTCCAGAGACAATTCCAAGAACACGCTGTATCTGCAAATGAACAGCCTGA | |
| GAGCCGAGGACACGGCTGTGTATTACTGTGCGAGAGGGGATGACTACGGTGA | |
| CTACCACCCCCACTACTACTACTACGGTTTGGACGTCTGGGGCCAAGGGACC | |
| ACGGTCACCGTCTCCTCA 3′ | |

Heavy chain variable region

Amino acid sequence

!5′QVQLVESGGGVVQPGRSLRLSCAASGFTFSTYGMHWVRQAPGKGLEWVTFIW? (SEQ ID NO: 2)? ? ! !YDGRNKYYADSVKGRFTVSRDNSKNTLYLQMNSLRAEDTAVYYCARGDDYGD? ! !YHPHYYYYGLDVWGQGTTVTVSS 3′? ! TABLE 10

43H7 Heavy chain V region domains.

| | | AA | |
|---|---|---|---|
| REGION | SEQUENCE | RESIDUES* | SEQ ID |
| FRI | QVQLVESGGVVQPGRSLRLSCAASGFTFS | 1-30 | SEQ ID NO: 3 |
| CDR1 | TYGMH | 31-35 | SEQ ID NO: 4 |
| FR2 | WVRQAPGKGLEWVT | 36-49 | SEQ ID NO: 5 |
| CDR2 | FIWYDGRNKYYADSVKG | 50-66 | SEQ ID NO: 6 |
| FR3 | RFTVSRDNSKNTLYLQMNSLRAEDTAVYYCAR | 67-98 | SEQ ID NO: 7 |
| CDR3 | GDDYGDYHPHYYYYGLDV | 99-116 | SEQ ID NO: 8 |
| FR4 | WGQGTTVTVSS | 117-127 | SEQ ID NO: 9 |

*AA Residues of SEQ ID NO: 2

Antibody-43H7
Light chain variable region

Nucleotide sequence

| | |
|---|---|
| 5′GATATTGTGATGACCCAGACTCCACTCTCTCTGTCCGTCACCCCTGGACAGC | (SEQ ID NO: 10) |
| CGGCCTCCATCTCCTGCAAGTCTAGTCAGAGCCTCCTGGATAGTGATGGAAA | |
| GACCTATTTGTATTGGTACCTGCAGAAGCCAGGCCAGCCTCCACAGCTCCTG | |
| ATCTATGAAGTTTCCAACCGGTTCTCTGGAGTGCCAGATAGGTTCAGTGGCAG | |
| CGGGTCAGGGACAGATTTCACACTGAAGATCAGCCGGGTGGAGGCTGAGGAT | |
| GTTGCATATTATTCTGCATGCAAAGTATACAGCTTCCGTGGACGTTCGGCCA | |
| AGGGACCAAGGTGGAAATCAAA 3′ | |

Light chain variable region

Amino acid sequence

| | |
|---|---|
| 5′DIVMTQTPLSLSVTPGQPASISCKSSQSLLDSDGKTYLYWYLQKPGQPPQLLIYE | (SEQ ID NO: 11) |
| VSNRFSGVPDRFSGSGSGTDFTLKISRVEAEDVAYYYCMQSIQLPWTFGQGTKVE | |
| IK3′ | |

TABLE 11

43H7 Light chain V region domains.

| | | AA | |
|---|---|---|---|
| REGION | SEQUENCE | RESIDUES* | SEQ ID |
| FRI | DIVMTQTPLSLSVTPGQPASISC | 1-23 | SEQ ID NO: 12 |
| CDR1 | KSSQSLLDSDGKTYLY | 24-39 | SEQ ID NO: 13 |
| FR2 | WYLQKPGQPPQLLIY | 40-54 | SEQ ID NO: 14 |
| CDR2 | EVSNRFS | 55-61 | SEQ ID NO: 15 |
| FR3 | GVPDRFSGSGSGTDFTLKISRVEAEDVAYYYC | 62-93 | SEQ ID NO: 16 |
| CDR3 | MQSIQLPWT | 94-102 | SEQ ID NO: 17 |
| FR4 | FGQGTKVEIK | 103-112 | SEQ ID NO: 18 |

*AA Residues of SEQ ID NO: 11

Antibody-42C1
Heavy chain variable region

Nucleotide sequence

| | |
|---|---|
| 5′CAGGTGCAGCTGCAGGAGTCGGGCCCAGGACTGGTGAAGCCTTCACAGAC | (SEQ ID NO: 19) |
| CCTGTCCCTCACCTGCACTGTCTCTGGTGTCTCCATCAGCAGTGGTGGTTACT | |
| ACTGGAGCTGGATCCGCCAGCACCCAGGGAAGGGCCTGGAGTGGATTGGGTA | |
| CATCTATTACAGTGGGAGCACCTACTACAACCCGTCCCTCAAGAGTCGAGTT | |
| ACCATATCAGTAGACACGTCTAAGAACCAGTTCTCCCTGAAGCTGAGCTCTGT | |
| GACTGCCGCGGACACGGCCGTGTTTTACTGTGCGAGAGAGGGGGAGTACTAC | |
| GATGAGGGGGGGTTTGACTACTGGGGCCAGGGAACCCTGGTCACCGTCTCCT | |
| CA 3′ | |

Heavy chain variable region

Amino acid sequence

| | |
|---|---|
| 5′QVQLQESGPGLVKPSQTLSLTCTVSGVSISSGGYYWSWIRQHPGKGLEWIGYIY | (SEQ ID NO: 20) |
| YSGSTYYNPSLKSRVTISVDTSKNQFSLKLSSVTAADTAVFYCAREGEYYDEGGF | |
| DYWGQGTLVTVSS 3′ | |

TABLE 12

42C1 Heavy chain V region domains.

| | | AA | |
|---|---|---|---|
| REGION | SEQUENCE | RESIDUES* | SEQ ID |
| FRI | QVQLQESGPGLVKPSQTLSLTCTVSGVSIS | 1-30 | SEQ ID NO: 21 |
| CDR1 | SGGYYWS | 31-37 | SEQ ID NO: 22 |
| FR2 | WIRQHPGKGLEWIG | 38-51 | SEQ ID NO: 23 |
| CDR2 | YIYYSGSTYYNPSLKS | 52-67 | SEQ ID NO: 24 |
| FR3 | RVTISVDTSKNQFSLKLSSVTAADTAVFYCAR | 68-99 | SEQ ID NO: 25 |
| CDR3 | EGEYYDEGGFDY | 100-111 | SEQ ID NO: 26 |
| FR4 | WGQGTLVTVSS | 112-122 | SEQ ID NO: 27 |

*AA Residues of SEQ ID NO: 20

Antibody-42C1
Light chain variable region

Nucleotide sequence

| | |
|---|---|
| 5′GAAATAGTGATGACGCAGTCTCCAGCCACCCTGTCTGTGTCTCCAGGGGAA | (SEQ ID NO: 28) |
| AGAGCCACCCTCTCCTGCAGGGCCAGTCAGAGTGTTAGCAGCAACTTAGCCT | |
| GGTACCAGCAGAAACCTGGCCAGGCTCCCAGGCTCCTCATCTATGGTGCATC | |
| CACCAGGGCCACTGGTATCCCAGCCAGGTTCAGTGGCAGTGGGTCTGGGACA | |
| GAGTTCACTCTCACCATCAGCAGCCTGCAGTCTGAAGATTTTGCAGTTTATTA | |
| CTGTCAGCAGTATAATAACTGGCCTCCGTGGACGTTCGGCCAAGGGACCAAG | |
| GTGGAAATCAAA 3′ | |

Light chain variable region

Amino acid sequence

| | |
|---|---|
| 5′EIVMTQSPATLSVSPGERATLSCRASQSVSSNLAWYQQKPGQAPRLLIYGASTR | (SEQ ID NO: 29) |
| ATGIPARFSGSGSGTEFTLTISSLQSEDFAVYYCQQYNNWPPWTFGQGTKVEIK 3′ | |

TABLE 13

42C1 Light chain V region domains.

| | | AA | |
|---|---|---|---|
| REGION | SEQUENCE | RESIDUES* | SEQ ID |
| FRI | EIVMTQSPATLSVSPGERATLSC | 1-23 | SEQ ID NO: 30 |
| CDR1 | RASQSVSSNLA | 24-34 | SEQ ID NO: 31 |
| FR2 | WYQQKPGQAPRLLIY | 35-49 | SEQ ID NO: 32 |
| CDR2 | GASTRAT | 50-56 | SEQ ID NO: 33 |
| FR3 | GIPARFSGSGSGTEFTLTISSLQSEDFAVYYC | 57-88 | SEQ ID NO: 34 |
| CDR3 | QQYNNWPPWT | 89-98 | SEQ ID NO: 35 |
| FR4 | FGQGTKVEIK | 99-108 | SEQ ID NO: 36 |

*AA Residues of SEQ ID NO: 29

Antibody-9G12
Heavy chain variable region

Nucleotide sequence

| | |
|---|---|
| 5′CAGGTGCAGCTGGTGGAGTCTGGGGGAGGCGTGGTCCAGCCTGGGAGGTC | (SEQ ID NO: 37) |
| CCTGAGACTCTCCTGTGCAGCGTCTGGATTCACCTTCAGTGGCTATGGCATGC | |
| ACTGGGTCCGCCAGGCTCCAGGCAAGGGGCTGGAGTGGGTGGCATTTATATG | |
| GTATAATGGAAATGATAATTACTATACAGACTCCGTGAAGGGCCGATTCACC | |
| ATCTCCAGAGACAATTCCAAGAACACGCTGTATCTGCAAATGAACAGCCTGA | |
| GAGCCGAGGACACGGCTGTGTATTACTGTGCGAGAGGGGATGACTACGGTGA | |
| CTACCACCCCCACTACTACTACTACGGTATGGACGTCTGGGGCCAAGGGACC | |
| ACGGTCACCGTCTCCTCA 3′ | |

Heavy chain variable region

Amino acid sequence

| | |
|---|---|
| 5′QVQLVESGGGVVQPGRSLRLSCAASGFTFSGYGMHWVRQAPGKGLEWVAFI | (SEQ ID NO: 38) |
| WYNGNDNYYTDSVKGRFTISRDNSKNTLYLQMNSLRAEDTAVYYCARGDDYG | |
| DYHPHYYYYGMDVWGQGTTVTVSS 3′ | |

TABLE 14

9G12 Heavy chain V region domains.

| | | AA | |
|---|---|---|---|
| REGION | SEQUENCE | RESIDUES* | SEQ ID |
| FRI | QVQLVESGGGVVQPGRSLRLSCAASGFTFS | 1-30 | SEQ ID NO: 39 |
| CDR1 | GYGMH | 31-35 | SEQ ID NO: 40 |
| FR2 | WVRQAPGKGLEWVA | 36-49 | SEQ ID NO: 41 |
| CDR2 | FIWYNGNDNYYTDSVKG | 50-66 | SEQ ID NO: 42 |
| FR3 | RFTISRDNSKNTLYLQMNSLRAEDTAVYYCAR | 67-98 | SEQ ID NO: 43 |
| CDR3 | GDDYGDYHPHYYYYGMDV | 99-116 | SEQ ID NO: 44 |
| FR4 | WGQGTTVTVSS | 117-127 | SEQ ID NO: 45 |

*AA Residues of SEQ ID NO: 38

Antibody-9G12
Light chain variable region

Nucleotide sequence

| | |
|---|---|
| 5′GATATTGTGATGACCCAGACTCCACTCTCTCTGTCCGTCACCCCTGGACAGC | (SEQ ID NO: 46) |
| CGGCCTCCATCTCCTGCAAGTCTAGTCAGAGCCTCCTGGATAGTGATGGAAA | |
| GACCTATTTGTATTGGTACCTGCAGAAGCCAGGCCAGCCTCCACAGCTCCTG | |
| ATCTATGAAGTTTCCAACCGGTTCTCTGGAGTGCCAGATAGGTTCAGTGGCAG | |
| CGGGTCAGGGACAGATTTCACACTGAAAATCAGCCGGGTGGAGGCTGAGGAT | |
| ATTGGGGTTTATTACTGCTTGCAAAGTATACAGCTTCCGTGGACGTTCGGCCA | |
| AGGGACCAAGGTGGAAATCAAA 3′ | |

Light chain variable region

Amino acid sequence

| | |
|---|---|
| 5′DIVMTQTPLSLSVTPGQPASISCKSSQSLLDSDGKTYLYWYLQKPGQPPQLLIYE | (SEQ ID NO: 47) |
| VSNRFSGVPDRFSGSGSGTDFTLKISRVEAEDIGVYYCLQSIQLPWTFGQGTKVEI | |
| K 3′ | |

TABLE 15

9G12 Light chain V region domains.

| | | AA | |
|---|---|---|---|
| REGION | SEQUENCE | RESIDUES* | SEQ ID |
| FRI | DIVMTQTPLSLSVTPGQPASISC | 1-23 | SEQ ID NO: 48 |
| CDR1 | KSSQSLLDSDGKTYLY | 24-39 | SEQ ID NO: 49 |
| FR2 | WYLQKPGQPPQLLIY | 40-54 | SEQ ID NO: 50 |
| CDR2 | EVSNRFS | 55-61 | SEQ ID NO: 51 |
| FR3 | GVPDRFSGSGSGTDFTLKISRVEAEDIGVYYC | 62-93 | SEQ ID NO: 52 |
| CDR3 | LQSIQLPWT | 94-102 | SEQ ID NO: 53 |
| FR4 | FGQGTKVEIK | 103-112 | SEQ ID NO: 54 |

*AA Residues of SEQ ID NO: 47

Antibody-35F1
Heavy chain variable region

Nucleotide sequence

| | |
|---|---|
| 5′CAGGTGCAGCTGCAGGAGTCGGGCCCAGGACTGGTGAAGCCTTCACAGAC | (SEQ ID NO: 55) |
| CCTGTCCCTCACCTGCACTGTCTCTGGTGGCTCCATCAAAAGTGGTGATTACT | |
| ACTGGAGCTGGGTCCGCCAGCTCCCAGGGAAGGGCCTGGAGTGGATTGGATA | |
| CATCTATTACAGTGGGAGCACCTACTACAACCCGTCCCTCAAGAGTCGAGTT | |
| ACCATATCAATAGACACGTCTAAGAACCAGTTCTCCCTGAAGCTGAGCTCTGT | |
| GACTGCCGCGGACACGGCCGTGTATTACTGTGCGAGAGAAGGGTCTTACTAT | |
| GATTCGGGAGGCTTTGACTACTGGGGCCAGGGAACCCTGGTCACCGTCTCCT | |
| CA 3′ | |

Heavy chain variable region

Amino acid sequence

| | |
|---|---|
| 5′QVQLQESGPGLVKPSQTLSLTCTVSGGSIKSGDYYWSWVRQLPGKGLEWIGYI | (SEQ ID NO: 56) |
| YYSGSTYYNPSLKSRVTISIDTSKNQFSLKLSSVTAADTAVYYCAREGSYYDSGG | |
| FDYWGQGTLVTVSS 3′ | |

TABLE 16

35F1 Heavy chain V region domains.

| | | AA | |
|---|---|---|---|
| REGION | SEQUENCE | RESIDUES* | SEQ ID |
| FRI | QVQLQESGPGLVKPSQTLSLTCTVSGGSIK | 1-30 | SEQ ID NO: 57 |
| CDR1 | SGDYYWS | 31-37 | SEQ ID NO: 58 |
| FR2 | WVRQLPGKGLEWIG | 38-51 | SEQ ID NO: 59 |
| CDR2 | YIYYSGSTYYNPSLKS | 52-67 | SEQ ID NO: 60 |
| FR3 | RVTISIDTSKNQFSLKLSSVTAADTAVYYCAR | 68-99 | SEQ ID NO: 61 |
| CDR3 | EGSYYDSGGFDY | 100-111 | SEQ ID NO: 62 |
| FR4 | WGQGTLVTVSS | 112-122 | SEQ ID NO: 63 |

*AA Residues of SEQ ID NO: 56

Antibody-35F1
Light chain variable region

Nucleotide sequence

| | |
|---|---|
| 5′GAAATAGTGATGACGCAGTCTCCAACCACCCTGTCTGTGTCTCCAGGGGAA | (SEQ ID NO: 64) |
| AGAGCCACCCTCTCCTGTAGGGCCGGTCAGAGTGTTGGCAACAACTTAGCCT | |
| GGTACCAGCAGAAACCTGGCCAGGCTCCCAGGCTCCTCATCTATGGTGCATC | |
| CACCAGGGCCACTGGTATCCCAGCCAGGTTCAGTGGCAGTGGGTCTGGGACA | |
| GAGTTCACTCTCACCATCAGCAGCCTGCAGTCTGAAGATTTTGCAGTTTATTA | |
| CTGTCAGCAGTATAATAACTGGCCTCCGTGGACGTTCGGCCAAGGGACCAAG | |
| GTGGAAATCAAC 3′ | |

Light chain variable region

Amino acid sequence

| 5′EIVMTQSPTTLSVSPGERATLSCRAGQSVGNNLAWYQQKPGQAPRLLIYGAST | (SEQ ID NO: 65) |
|---|---|
| RATGIPARFSGSGSGTEFTLTISSLQSEDFAVYYCQQYNNWPPWTFGQGTKVEIN | |
| 3′ | |

TABLE 17

35F1 Light chain V region domains.

| | | AA | |
|---|---|---|---|
| REGION | SEQUENCE | RESIDUES* | SEQ ID |
| FRI | EIVMTQSPTTLSVSPGERATLSC | 1-23 | SEQ ID NO: 66 |
| CDR1 | RAGQSVGNNLA | 24-34 | SEQ ID NO: 67 |
| FR2 | WYQQKPGQAPRLLIY | 35-49 | SEQ ID NO: 68 |
| CDR2 | GASTRAT | 50-56 | SEQ ID NO: 69 |
| FR3 | GIPARFSGSGSGTEFTLTISSLQSEDFAVYYC | 57-88 | SEQ ID NO: 70 |
| CDR3 | QQYNNWPPWT | 89-98 | SEQ ID NO: 71 |
| FR4 | FGQGTKVEIN | 99-108 | SEQ ID NO: 72 |

*AA Residues of SEQ ID NO: 65

The variable heavy chain nucleotide sequences were analyzed to determine the VH family, the D-region sequence and the J-region sequence. The sequences were then translated to determine the primary amino acid sequence and compared to the germline V_{H}, D and J-region sequences to assess somatic hypermutations. The selected germline sequences of anti-SLPI antibodies are shown in Table 18.

TABLE 18
Anti-SLPI antibody germline

| | |
|---|---|
| Heavy Chain | Light Chain |

| Antibody | VH | D | J | V kappa | J |
|---|---|---|---|---|---|
| 42C1 | VH4-31 | D2-2 | JH4b | L2VK3 | JK1 |
| 35F1 | VH4-31 | D3-10 | JH4b | L2VK3 | JK1 |
| 43H7 | VH3-33 | D4-17 | JH6b | A2VK2 | JK1 |
| 9G12 | VH3-33 | D3-16 | JH6b | A2VK2 | JK1 |

The primary amino acid sequences of the anti-SLPI antibody heavy chains V regions are shown above in Tables 10, 12, 14 and 16. The light chain V regions amino acid sequences are shown above in Tables 11, 13, 15 and 17.

### Example 8

### Domain Analysis of Anti-SLPI antibodies

The variable (V) regions of immunoglobulin chains are encoded by multiple germline DNA segments, which are joined into functional variable regions (V_{H}DJ_{H} or V_{K}J_{K}) during B-cell ontogeny. The molecular and genetic diversity of the antibody response to SLPI was studied in detail. Analysis of four individual antibodies specific to SLPI showed preference for two germline VH genes, V_{H}4-31 and V_{H}3-33 (Table 18). In addition, the V_{H}4-31 germline genes were preferentially paired with the J_{H}4b germ line J region and the V_{H}3-33 germline genes were preferentially paired with the J_{H}6b germline J region. As shown in Table 18, antibodies against SLPI showed a preference for the L2V_{K}3 and A2V_{K}2 light chain germline genes, both preferentially paired with J_{K}1 light chain germline gene.

Epitope Binning and BiaCore® Affinity Determination

Epitope Binning

Certain antibodies, described herein are “binned” in accordance with the protocol described in U.S. patent application Publication No.20030157730 MxhIgG conjugated beads are prepared for coupling to primary antibody. The volume of supernatant needed is calculated using the following formula: (n+10)×50 μL (where n=total number of samples on plate). Where the concentration is known, 0.5 μg/mL is used. Bead stock is gently vortexed, then diluted in supernatant to a concentration of 2500 of each bead per well or 0.5×105/mL and incubated on a shaker in the dark at RT overnight, or 2 hours if at a known concentration of 0.5 μg/mL. Following aspiration, 50 μL of each bead is added to each well of filter plate, then washed once by adding 100 μL/well wash buffer and aspirating. Antigen and controls are added to filter plate 50 μL/well then covered and allowed to incubate in the dark for 1 hour on shaker. Following a wash step, a secondary unknown antibody is added at 50 μL/well using the same dilution (or concentration if known) as is used for the primary antibody. The plates are then incubated in the dark for 2 hours at RT on shaker followed by a wash step. Next, 50 μL/well biotinylated mxhIgG diluted 1:500 is added and allowed to incubate in the dark for 1 hour on shaker at RT. Following a wash step, 50 μL/well Streptavidin-PE is added at 1:1000 and allowed to incubate in the dark for 15 minutes on shaker at RT. Following a wash step, each well is resuspended in 80 μL blocking buffer and read using Luminex. Results show that the monoclonal antibodies belong to distinct bins. Competitive binding by antibodies from different bins supports antibody specificity for similar or adjacent epitopes. Non competitive binding supports antibody specificity for unique epitopes.

Determination of Anti-SLPI mAb Affinity using BiaCore® Analysis

BiaCore® analysis was used to determine binding affinity of anti-SLPI antibody to SLPI antigen. The analysis was performed at 25° C. using a BiaCore® 2000 biosensor equipped with a research-grade CM5 sensor chip. A high-density goat α human antibody surface over a CM5 BiaCore® chip was prepared using routine amine coupling. Antibody supernatents were diluted to ˜5 μg/mL in HBS-P running buffer containing 100 μg/mL BSA and 10 mg/mL carboxymethyldextran. The antibodies were then captured individually on a separate surface using a 2 minute contact time, and a 5 minute wash for stabilization of antibody baseline.

SLPI antigen was injected over each surface for 75 seconds, followed by a 3-minute dissociation. Double-referenced binding data were obtained by subtracting the signal from a control flow cell and subtracting the baseline drift of a buffer inject just prior to the SLPI injection. SLPI binding data for each mAb were normalized for the amount of mAb captured on each surface. The normalized, drift-corrected responses were also measured. The kinetic analysis results of anti-SLPI mAB binding at 25° C. are listed in Table 19 below.

TABLE 19
KDS for SLPI-specific mAbs

| Antibody | Affinity pM by BIAcore |
|---|---|
| 43H7 | 104 |
| 42C1 | 116 |
| 9G12 | 53 |
| 35F1 | 140 |

### Example 9

### Elastase Inhibition Assay

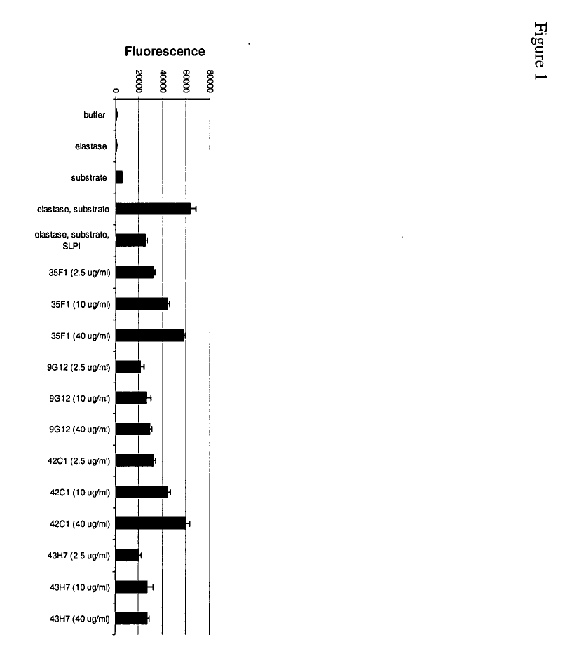

Recombinant human SLPI (R&D Systems, Minneopaolis, Minn.) (stock conc. 1 mg/ml: diluted to 100 μg/ml, 10 μg/ml, 1 μg/ml; 4 μl/well) was mixed with each of the monoclonal antibodies to result in final SLPI concentrations of 40, 10, 2 μg/ml and incubated at RT for 5 min. Human elastase (Calbiochem, La Jolla, Calif.)at 0.6 μ/well (50 μg reconstituted in 169 μl buffer:50 mM NaOAc, pH 5.5, 200 mM NaCl, final concentration 10 μM) was mixed with 0 (control) or 2 μl of each SLPI dilution, or SLPI mixed with antibody in working buffer (final volume of 100 μl). A control was also prepared using just 100 μl working buffer. The samples were incubated at 37° C. for 15 min. 100 μl of BODIPY substrate was added and the samples were incubated at RT for at least 60 min (incubation at 37° C. overnight can enhance the detectable signal), protected from light. The samples were then read by CytoFluo fluorometer (excitation=480±25 nm, emission=530±25 nm). Results are shown in FIG. 1. Monoclonal antibody 35F1 is referred to as #50; 42C1 is referred to as #24; 43H7 is referred to as #11; and 9G12 is referred to as #36. Dose dependent inhibition of SLPI-elastase activity was demonstrated with each antibody. Antibodies 35F1 and 42C1 showed the greatest neutralization activity in this assay.

### Example 10

### Cathepsin G Inhibition Assay

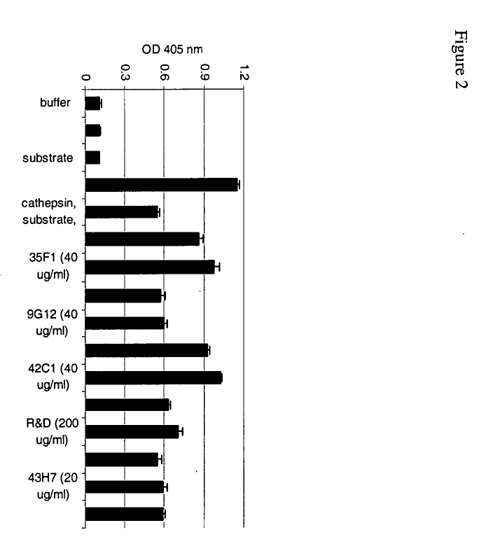

2 μl of recombinant human SLPI diluted to 100 μg/ml, 10 μg/ml, 1 μg/ml, was added to each of the monoclonal antibodies to result in a final concentration of 40, 10, 2 μg/ml and incubated at RT for 5 min. 5 μl of Cathepsin G (Calbiochem La Jolla, Calif.) (100 mU Cathepsin G reconstituted in 100 μl buffer containing 50 mM NaOAc, pH5.5, 150 mM NaCl; final conc. 1 mU/μl) was mixed with 0 (control), 2 μl diluted SLPI, or SLPI with antibody in working buffer (100 mM Tris-HCl, pH8.3, 0.96 M NaCl, 1%BSA), final volume of 98 μl. A control was also prepared using just 100 μl working buffer. Samples were incubated at 37° C. for 15 min, then 2 μl of substrate was added and incubated at RT for at least 30 min, protected from light. The samples were read at OD 405 nm. Results are shown in FIG. 2. Monoclonal antibody 35F1 is referred to as #50; 42C1 is referred to as #24; 43H7 is referred to as #11; and 9G12 is referred to as #36. Dose dependent inhibition of SLPI-Cathepsin G activity was demonstrated using antibody 35F1, 42C1 and 9G12. Antibodies 35F1 and 42C1 had the greatest effect.

### Example 11

### IHC Evaluation on Human Oncology Tissue MicroArray

Monoclonal antibodies 35F1, 42C1 and 9G12 were evaluated for reactivity with frozen and fixed positive control OVCAR-8 ovarian carcinoma cells; IGROV-1 xenograft tissue (Institute for Drug Developent, San Antonio, Tex.); human ovarian cancer tissue specimens tissue (NDRI); human oncology tissue microarrays (TMA, from Ardais, Lexington Mass.); or human tissue arrays obtained from Biogenix (San Ramon, Calif.), by immunohistochemistry (IHC). As antibody 42C1 stained fixed OVCAR-8 cells, this antibody was selected for evaluation of reactivity on other tissue specimens. Controls included isotype matched antibody PK16.3 and a positive control ovarian carcinoma specimen.

Tissue sections (5 μm) were cut from formalin fixed and paraffin embedded tissue samples derived from either IGROV-1 xenograft or human ovarian cancer tissue. These sections were stained with 42C1 antibody by one of two methods, depending upon the source of the material. In both cases, tissue sections were rehydrated through incubations in xylene and a graded series of ethanols terminating in PBS. Also, in both cases, endogenous peroxidase activity was quenched in a 3% solution of hydrogen peroxide in methanol.

Staining of xenograft tissue was performed as follows: tissue sections were blocked in blocking buffer (5% BSA (Sigma), 1% goat serum (Jackson Immunolabs, West Grove, Pa.) in PBS) for 1 hour. Sections were incubated with purified 42C1 antibody or isotype control (IgG2; Fitzgerald Industries) diluted in blocking buffer. After 1 hour, ections were washed in 3 changes of PBS for 5 to 10 minutes each. The sections were then incubated for 45 minutes with a 1:200 dilution of biotinylated goat anti-human IgG (Jackson Immunolabs), diluted in blocking buffer. Sections were washed and incubated with a 1:200 dilution of streptavidin conjugated horseradish peroxidase (Jackson Immunolabs) in blocking buffer for 30 minutes and then washed as before. Antibody was detected using DAB reagent (Vector labs, CITY STATE). Sections were counterstained in hematoxylin (Fisher Scientific) and dehydrated through alcohol and xylene and coverslipped with permount (Fisher Scientific).

Staining of human tissue samples followed essentially the same protocol, except that the primary and secondary antibodies were precomplexed in 5% BSA and 1% goat serum in PBS for 1 hour at 37° C. at a molar ratio of approximately 10:1 of 42C1 or control IgG to secondary biotinylated goat anti-human antibody. The complexes were then blocked with a 1:2000 dilution of human serum and incubated again for 1 hour at 37° C. The complexes were applied for 1 hour to tissue sections that had been processed as above to the protein-blocking step and staining completed as described above. IHC results obtained with Ardais microarray tissues are shown in Table 20.

TABLE 20
Immunohistochemistry staining results

| | | | | | | High and Moderate | Low |
|---|---|---|---|---|---|---|---|
| | Spot | Score | Score | Score | Score | Expression | Expression |
| Name of Tissue | Amount | 0 | 1 | 2 | 3 | Frequency | Frequency |
| Brain Cancer | 15 | 12 | 3 | 0 | 0 | 0 | 20 |
| Breast Cancer | 20 | 13 | 6 | 1 | 0 | 5 | 30 |
| Colon Cancer | 19 | 13 | 6 | 0 | 0 | 0 | 31.6 |
| Endometrial Cancer | 19 | 4 | 4 | 7 | 4 | 57.9 | 21.1 |
| Kidney Cancer | 20 | 17 | 1 | 2 | 0 | 10 | 5 |
| Lung Cancer | 18 | 7 | 9 | 2 | 0 | 11.1 | 50 |
| Lymphoma | 20 | 11 | 8 | 1 | 0 | 5 | 40 |
| Malignant Melanoma | 19 | 14 | 5 | 0 | 0 | 0 | 26.3 |
| Ovarian Carcinoma | 18 | 6 | 7 | 5 | 0 | 27.8 | 38.9 |
| Prostate Cancer | 20 | 18 | 2 | 0 | 0 | 0 | 10 |
| Lymph Node | 2 | 0 | 2 | 0 | 0 | 0 | 100 |
| Normal Breast | 9 | 6 | 3 | 0 | 0 | 0 | 33.3 |
| Normal Colon | 10 | 10 | 0 | 0 | 0 | 0 | 0 |
| Normal Endometrium | 10 | 4 | 5 | 1 | 0 | 10 | 50 |
| Normal Kidney | 10 | 10 | 0 | 0 | 0 | 0 | 0 |
| Normal Liver | 4 | 4 | 0 | 0 | 0 | 0 | 0 |
| Normal Lung | 9 | 1 | 7 | 1 | 0 | 11.1 | 77.8 |
| Normal Ovary | 8 | 8 | 0 | 0 | 0 | 0 | 0 |
| Normal Pancreas | 3 | 0 | 1 | 2 | 0 | 66.7 | 33.3 |
| Normal Prostate | 10 | 9 | 1 | 0 | 0 | 0 | 10 |
| Normal Salivary gland | 4 | 0 | 2 | 2 | 0 | 50 | 50 |
| Normal Spleen | 4 | 0 | 4 | 0 | 0 | 0 | 100 |
| Normal Testis | 4 | 3 | 1 | 0 | 0 | 0 | 25 |
| Normal Thyroid | 4 | 4 | 0 | 0 | 0 | 0 | 0 |
| Normal Tonsil | 3 | 2 | 1 | 0 | 0 | 0 | 33.3 |

0 (negative) no staining,
1 (weak) 1-100% of specific staining cells with 1+ staining intensity or 1-20% of specific staining cells with 2+ staining intensity.
2 (moderate) 2+ staining intensity in 21-79% of specific staining cells or a 3+ staining intensity in 1-49% of specific staining cells.
3 (strong): 2+ staining intensity in 80-100% of specific staining cells or 3+ staining intensity in ≧50% of specific staining cells.
Frequency % of high and medium expression = (score 3 + score 2)/(Spot number) × 100%
Frequency % of low expression = score1/(Spot number) × 100%

Antibody 42C1 stained cancer specimens with high and moderate expression frequency observed at 57.9% in endometrial cancer, 27.8% in ovarian cancer, 11.1% in lung cancer, 10% in kidney cancer and 5% in breast cancer. In normal tissue specimens, high and moderate expression frequency was observed at 66.7% in pancreas, 50% in salivary glands, 11.1% in lung and 10% in endometrium. Weak staining was observed in colon cancer, breast cancer, brain cancer, melanoma, prostate cancer and lymphoma. Weak staining was observed on numerous normal and normal matched tissues, such as tonsil, lymph node, spleen and prostate.

Ovarian cancer tissue array (Biogenix) stained with 42C1 showed positive staining of SLPI cancer tissues from patients including: 3 of 6 adenomas, 3 of 4 adenocarcinomas and 6 of 10 poorly differentiated adenocarcinomas. None of 6 normal ovarian tissue samples were positively stained by 42C1.

### Example 12

### Western Blot Analysis

Mammalian tumor-derived cell lines, OVCAR-3, OVCAR-4, OVCAR-5, OVCAR-8, HT29, IGROV-1, SK-OV-3, TK-10, A498, Caki-2, MDAMB231, 786-0, U87MG, A549, SW480, SW620, MCF7,and A2780 obtained from the ATCC (Manassas, Va.) were grown in serum-free DMEM media for 3 days. Media was collected and immunoprecipitated by incubating with human anti-SLPI antibody (5 ug/ml) of the invention (42C1) and Protein Sepharose A beads (Pharmacia Mississauga, Canada), for 4 hrs. Beads were washed 5 times in IP buffer (150 mM NaCl, 50 mM Tris (pH 7.5), 1% NP-40), followed by denaturing at 95 C in western blot loading dye (Invitrogen Carlsbad, Calif.). The eluted proteins were resolved by SDS-polyacrylamide gel electrophoresis and blotted to nitrocellulose membrane. Blots were incubated with human anti-SLPI antibody for 24 hr at 4 C. The membrane was washed and probed with peroxidase-conjugated donkey anti-human IgG (H+L) (Jackson Immunolabs, West Grove, Pa.) at 1:1000 dilution for 1 hr at RT. Proteins were visualized by chemiluminescent detection.

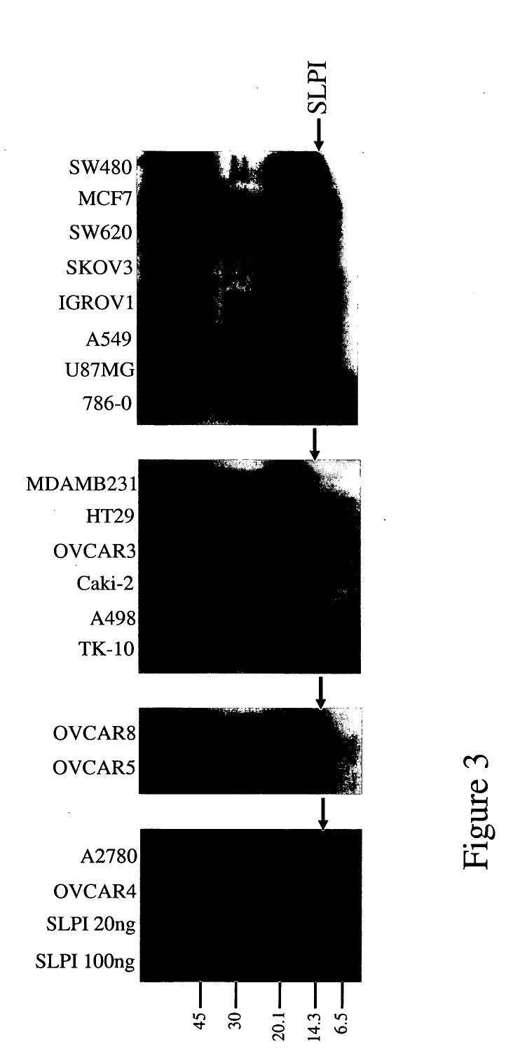

Using immunoprecipitation and western blot analysis SLPI protein expression was detected in the conditioned media of ovarian carcinoma cells, OVCAR-3, OVCAR-4, OVCAR-8 and IGROV-1; kidney carcinoma cell lines Caki-2, A498, 786-0; lung carcinoma cell line A549; colon carcinoma cell line HT29, SW480, SW620; and breast carcinoma cell line MCF7. Expression level was undetectable in ovarian cell lines OVCAR5, SKOV-3; as well as TK-10 (kidney), MDAMB231(breast), U87MG(glioblastoma) and A2780(lung) (FIG. 3).

### Example 13

### SLPI sera ELISA

A sandwich ELISA was developed to quantify SLPI levels in conditioned media collected from ovarian cancer cells and in serum of ovarian cancer patients using 42C1 antibody and goat anti-SLPI polyclonal antibody (R&D Systems, Minneapolis, Minn.) according to the following protocol:

50 μl of capture antibody goat anti-SLPI polyclonal antibody in coating buffer (0.1 M NaHCO₃, pH 9.6) at a concentration of 5 μg/ml was coated on ELISA plates and incubated at 4° C. overnight. Plates were then treated with 200 μl of blocking buffer (0.5% BSA, 0.1% Tween 20, 0.01% Thimerosal in PBS) for 1 hr at 25° C. Plates were washed (3×) using 0.05% Tween 20 in PBS washing buffer (WB). Conditioned media from ovarian cancer cells or normal or ovarian cancer patient sera (Clinomics, Bioreclamation, Cooperative Human Tissue Network) diluted 50% in blocking buffer. were incubated on the plates with for 2 hr at 25° C. Plates were washed with WB, and then incubated with 42C1 antibody (4 μg/ml) for 1 hr at 25° C. After washing, plates were incubated with secondary peroxidase-conjugated donkey anti-human antibody (SOURCE) for 1 hr, washed as before, and then treated with 100 μl/well of TMB substrate (Pharmingen). The reaction was stopped with 2M H₂SO₄ and analyzed using an ELISA plate reader at 450 nm with a correction of 550 nm. The concentration of SLPI was calculated by comparison to a SLPI standard curve using a four parameter curve fitting program.

In OVCAR-3, IGROV-1 and SK-OV-3 cells, the secrected SLPI level was 241 ng/ml/10⁶ cells, 202.8 ng/ml/10⁶ cells and 1.2 ng/ml/10⁶ cells, respectively, which corresponded with the western blot analysis. Increased level of SLPI was also detected in ovarian cancer patients (250.1 ng/ml) compared to that of normal volunteer (189.4 ng/ml)

### Example 14

### FACS Analysis of SLPI Expression

FACS analysis of anti-SLPI antibody binding to OVCAR4, OVCAR-5, OVCAR-8, SK-OV-3, A2780, SF294, 786-0 cell lines was done as follows. Suspended cells were washed twice with ice-cold FACS buffer (SOURCE) and incubated with antibody 42C1 (170 nM/FACS buffer) for 1 hr then washed. Cells were incubated with a 1:500 dilution in FACS buffer of peroxidase-conjugated donkey anti-human IgG (H+L) (Jackson Immunolabs) for 30 minutes. Cells were washed and then fixed with 1% formaldehyde in PBS. Analysis was done using a FACS Calibur™ flow cytometer (Becton Dickinson, Frankin Lakes, N.J.).

42C1 antibody bound SLPI on SLPI expressing ovarian cancer cell lines: OVCAR-4 (Geo Mean Ratio 37), OVCAR-5 (Geo Mean Ratio 10), and OVCAR-8 (Geo Mean Ratio 35) as compared to SLPI negative cell line 786-0 (Geo Mean Ratio 3).

### Example 15

### Effect of SLPI-Neutralizing Antibody on Ovarian Carcinoma Cells

The effect of SLPI on ovarian carcinoma cell proliferation, and the effect of a SLPI-neutralizing antibody on SLPI mediated cell proliferation was investigated.

OVCAR-3 ovarian carcinoma cells were plated in DMEM with 10% FBS in 96 well flat bottom plates at 1000 cells/well. Twenty-four hours later, 1)recombinant SLPI (0, 42.5, 85, 170, 340 and 680 nM in 100 μl DMEM media containing 10% FBS); 2) anti-SLPI antibody at concentrations ranging from 0 to 240 nM; 3) IgG control antibody; or 4) recombinant SLPI (70 nM) mixed with increasing concentrations of 42C1 antibody was added to the cells. After 72 hr media was removed, and 50 μl of trypsin was added to each well. Once cells were completely detached, 50 μl of growth media was added and mixed. Then, 20 μl of cell mixture was transferred to a 100 mm tissue culture dish. Cells were incubated at 37° C. for 7 days until colonies were formed. Colonies were stained using crystal violet solution and counted. The data was presented as % of untreated control.

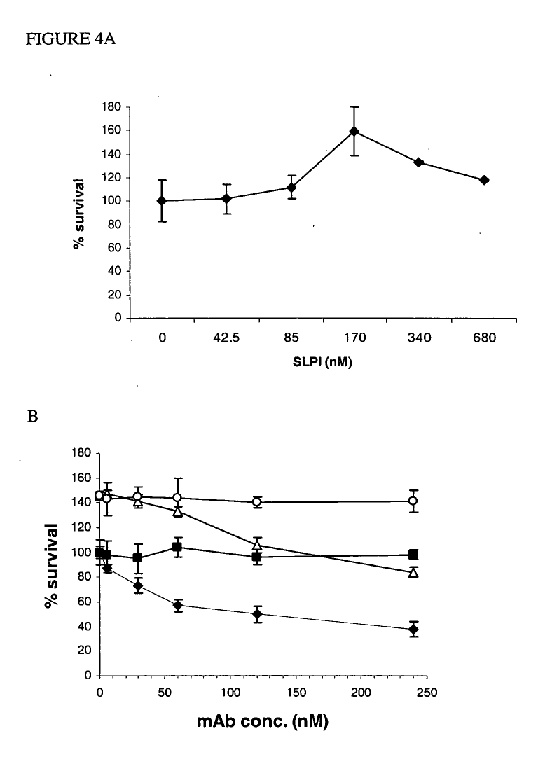

Exogenous SLPI increased OVCAR-3 cell proliferation by 50%, with an optimal dose of 170 nM (FIG. 4A). In the absence of exogenous SLPI, antibody 42C1 decreased OVCAR-3 cell proliferation in a dose dependent manner with maximum effect (60%) at 240 nM. Antibody 42C1 blocked OVCAR-3 cell proliferation induced by exogenously added SLPI protein (FIG. 4B). Furthermore, antibody 42C1 at 240 nM completely abolished the stimulatory effect of SLPI protein (170 nM) (SLPI:Antibody molar ratio approximately 1:1).

IGROV-1 carcinoma cells were plated in DMEM with 10% FBS in 96 well flat bottom plates at 1000 cells/well. Twenty-four hours later anti-SLPI antibodies at concentrations ranging from 0 to 240 nM or IgG control antibody was added to the cells. After 72 hr media was removed, and 50 μl of trypsin was added to each well. Once cells were completely detached, 50 μl of growth media was added and mixed. Then, 20 μl of cell mixture was transferred to a 100 mm tissue culture dish. Cells were incubated at 37° C for 7 days until colonies were formed. Colonies were stained using crystal violet solution and counted. The data was presented as % of untreated control.

Antibodies 35F1 and 42C1 inhibited the growth of IGOV-1 cells compared to isotype matched antibody treated cells, with an IC50 of 163 nM and 121 nM respectively. Both antibodies also inhibited the growth of SW480 in a similar assay with an IC50 of 85 nM

### Example 16

### Anti-SLPI Antibody Reversal of SLPI Inhibition of Elastase Activity on Cells

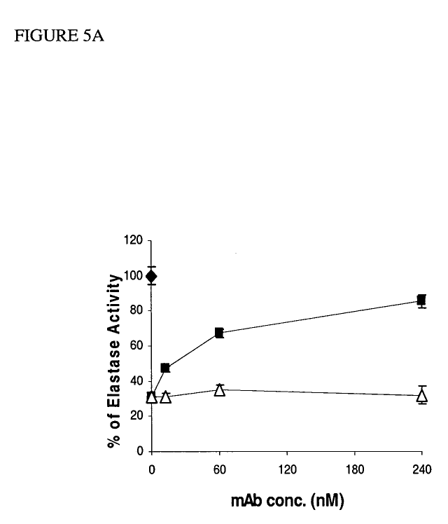

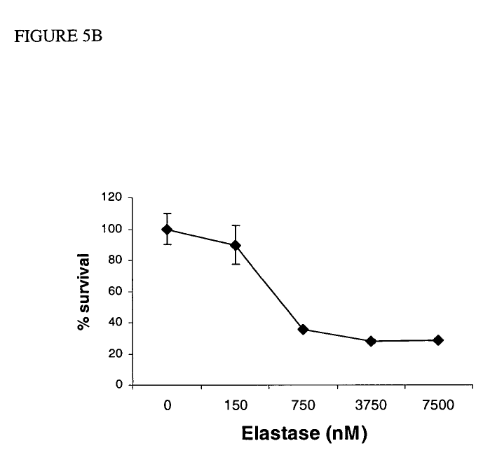

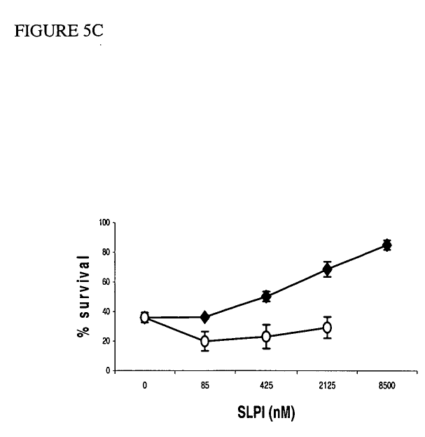

In a cell free assay, elastase (30 nM) was mixed with or without SLPI (170 nM) and with various concentrations of 42C1, incubated at 37° C. for 15 minutes. The substrate was then added to the mixture, and protease activity was detected by an increase in fluorescence. SLPI at a dose of 170 nM, inhibited elastase activity by 70% and 42C1 reversed SLPI mediated inhibition in a dose dependent manner (FIG. 5A).

Having observed that SLPI directly inhibited elastase activity, we -examined whether SLPI can rescue OVCAR-3 cells from elastase mediated toxicity. OVCAR-3 cells were plated in 96 well flat bottom plates at 2000 cells/well. Twenty-four hours later, 1) elastase (concentrations ranging from 150 to 7500 nM in 100 μl DMEM media containing 1% FBS); 2) elastase (750 nM) mixed with SLPI (concentrations ranging from 0-8500 nM); or 3) elastase (750 nM), SLPI (0-8500 nM) and anti-SLPI antibody (concentrations ranging from 0-2125 nM) mixture was added to the cells After 48 hr Titer-Blue™ cell viability assay was performed according to the manufacturer's specification (Promega). Elastase decreased cell viability in a dose dependent manner (FIG. 5B) and SLPI reversed this effect of elastase on the cells (FIG. 5C). 42C1 antibody inhibited the effect of SLPI rescue of cells from elastase toxicity (FIG. 5 C).

### Example 17

### In vivo Efficacy of Anti-SLPI Antibodies

SW480 colorectal adenocarcinoma xenografts were established by subcutaneous implantation of 30-40 mg of fragments of SW480 tumor in female nude mice. Dosing began on Day 1 in groups of ten mice bearing established (˜114 mm3) tumors. Anti-SLPI antibody 42C1 therapy was administered i.v. at 10, 3, and 1 mg/kg, given once every four days for a total of 4 doses (q4d×4). The 3 mg/kg of 42C1 regimen was also combined with a standard i.p. irinotecan (CPT) treatment, 100 mg/kg, once weekly for three weeks (qwk×3). A reference group received irinotecan monotherapy.

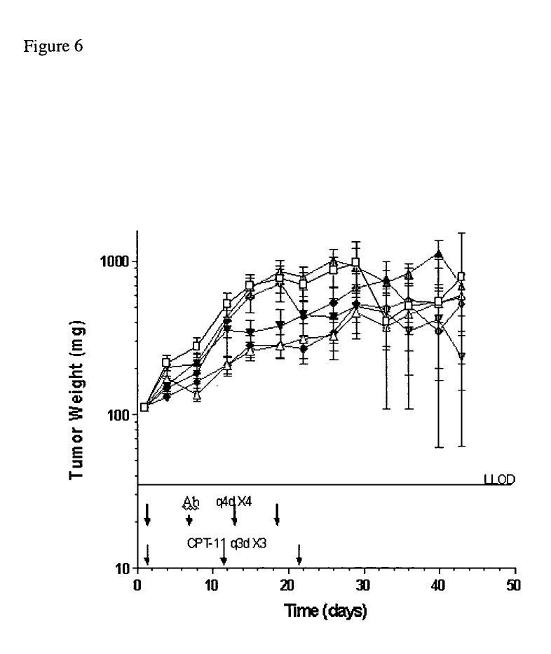

Anti-SLPI antibody therapy produced 80%, 33% and 4% tumor growth delay (%TGD) (defined as the percent increase in the median time to endpoint of drug-treated versus vehicle-treated mice) at 10, 3 and 1 mg/kg, respectively. At 10 mg/kg, antibody treatment yielded four 89-day survivors with a mean tumor volume (MTV) of 20 mm³ and two long term tumor free survivors (LTTFS); at 3 mg/kg there was one LTTFS; and at 1 mg/kg there were no survivors. Irinotecan therapy produced 118% TGD and no 89-day survivors. The combination of irinotecan with 3 mg/kg antibody produced 113% TGD, two 89-day survivors with a MTV of 198 mm³, and one partial regression (PR) response. All treatments were well tolerated. Results are shown in FIG. 6.

OVCAR-3 ovarian carcinoma xenografts are established by subcutaneous implantation of 30-40 mg fragments of OVCAR-3 tumor in female nude mice. Dosing began on Day 1 in groups of ten mice bearing established tumors (˜115 mm3). Anti-SLPI antibody 42C1 therapy is administered i.v. at 10, 3, and 1 mg/kg given once every four days for a total of 4 doses (q4d×4). The 3 mg/kg of Mab 42C1 regimen is also combined with i.v. paclitaxel treatment, 15 mg/kg (q2d×5). A reference group received paclitaxel monotherapy (15 mg/kg, q2d×5). Control mice received i.v. phosphate-buffered saline (vehicle). The tumor measurements and body weights are recorded twice weekly throughout the study period. The endpoint volume for tumor growth in the ten animal groups is 1200 mm³.

Results show treatment with Mab 42C1 reduces MTV, increases the number of animals surviving the study period and increases the number of LTTFS as compared to control mice receiving vehicle treatment alone.

### Example 18

### Diagnosing Cancer with Antibodies Against SLPI

A subject suspected of having a ovarian cancer tumor is identified and a tissue sample from the suspected tumor is removed for testing. The removed tissue is then contacted with anti-SLPI antibodies having a colorimetric label. A determination is made of whether the anti-SLPI antibodies bind specifically to the removed tissue. Binding is indicative of cancereous tissue while the absense of binding is indicative of non-cancerous tissue. The patient's conditition is diagnosed accordingly to facilitate subsequent testing, counseling, and/or treatment.

### Example 19

### Treating Cancer with Antibodies Against SLPI

Modulating SLPI activity is useful to treat a subject at risk for or afflicted with cancer. Such a subject would benefit from treatment with an anti-SLPI antibody of the present invention. Typically, antibodies are administered in an outpatient setting by weekly administration at about 0.1-1.0 mg/kg dose by slow intravenous (IV) infusion. The appropriate therapeutically effective dose of an antibody is selected by a treating clinician and would range approximately from 1 ug/kg to 20 mg/kg, from 1 ug/kg to 10 mg/kg, from 1 ug/kg to 1 mg/kg, from 10 ug/kg to 1 mg/kg, from 10 ug/kg to 100 ug/kg, from 100 ug/kg to 1 mg/kg, and from 500 ug/kg to 5 mg/kg.

The antibodies are also used to prevent and/or to reduce severity and/or symptoms of disease associated with SLPI activity.

To test the clinical efficacy of antibodies in humans, individuals with cancer, particularly, but not limited to ovarian, lung or colon carcinoma are identified and randomized into treatment groups. Treatment groups include a group not receiving antibody treatment and groups treated with different doses of anti-SLPI antibody. Individuals are followed prospectively and individuals receiving antibody treatment exhibit an improvement in their condition.

### Equivalents

The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. An isolated human antibody that specifically binds to SLPI.

2. The antibody of claim 1 in which the antibody modulates the activity of SLPI.

3. The antibody of claim 1 in which the antibody neutralizes the activity of SLPI.

4. The antibody of claim 1 in which the antibody is a monoclonal antibody.

5. The antibody of claim 1 in which the antibody specifically binds SLPI with an affinity constant greater than 10⁷M⁻¹.

6. The antibody of claim 4 comprising a region selected from the group comprising: VH4-31, VH3-33, L2VK3, and A2VK3. The antibody of claim 4 comprising a region selected derived from the group comprising: VH4-31, VH3-33, L2VK3, and A2VK3.

7. The antibody of claim 6 comprising the regions or regions derived from VH4-31 and L2VK3.

8. The antibody of claim 6 comprising the regions or regions derived from VH3-33 and A2VK3.

9. The antibody of claim 6 comprising an amino acid sequence as set out in SEQ ID NO:73, SEQ ID NO:74, SEQ ID NO:75 or SEQ ID NO:76.

10. The antibody of claim 6 comprising an amino acid sequence as set out in SEQ ID NO:2, SEQ ID NO:20, SEQ ID NO:38, SEQ ID NO:56, SEQ ID NO:73, SEQ ID NO:74, SEQ ID NO:82 or SEQ ID NO:84.

11. The antibody of claim 6 comprising an amino acid sequence as set out in SEQ ID NO:11, SEQ ID NO:29, SEQ ID NO:47, SEQ ID NO:65, SEQ ID NO:75; SEQ ID NO:76, SEQ ID NO: 106, or SEQ ID NO: 108.

12. The antibody of claim 6 comprising an amino acid sequence as set out in SEQ ID NO:8, SEQ ID NO:26, SEQ ID NO:44; or SEQ ID NO:62.

13. The antibody of claim 6 wherin the antibody is 43H7, 42C1, 9G12 or 35F1.

14. A pharmaceutical composition comprising the antibody of claim 1.

15. A method of treatment comprising administering a therapeutically effective dose of the pharmaceutical composition of claim 15.

16. The method of claim 16, wherein the pharmaceutical composition is administered to a subject in need of treatment or prevention of cancer.

17. The method of claim 16, wherein the subject is human.

18. An antibody comprising human framework regions and means for specific binding to SLPI.

19. The antibody of claim 19, wherein the antibody is capable of inhibiting elastase activity.

20. The antibody of claim 19, wherein the means comprises a CDR derived from 43H7, 42C1, 9G12 or 35F1.

21. An isolated nucleic acid encoding the antibody of claim 1.

22. An expression vector comprising the nucleic acid of claim 22.

23. A host cell comprising the vector of claim 23.

24. The host cell of claim 24, wherein the host cell is chosen from: an E. coli bacterium, a Chinese hamster ovary cell, a HeLa cell, and a NSO cell.

25. The nucleic acid of claim 22, wherein the nucleic acid encodes the amino acid sequence set out in SEQ ID NO:2, SEQ ID NO: 11, SEQ ID NO:20, SEQ ID NO:29, SEQ ID NO:38, SEQ ID NO:47, SEQ ID NO:65, SEQ ID NO:65, SEQ ID NO:73, SEQ ID NO:74, SEQ ID NO:75, SEQ ID NO:76, SEQ ID NO:82, SEQ ID NO:84, SEQ ID NO: 106, or SEQ ID NO: 108.

26. The nucleic acid of claim 26, wherein the nucleic acid comprises a nucleotide sequence selected from the group consisting of SEQ ID NO: 1, SEQ ID NO: 10, SEQ ID NO: 19, SEQ ID NO:28, SEQ ID NO:37, SEQ ID NO:46, SEQ ID NO:55, SEQ ID NO:64, SEQ ID NO:81, SEQ ID NO:83, SEQ ID NO:105, or SEQ ID NO: 107.

27. A method of making a human monoclonal antibody that specifically binds with SLPI, the method comprising the steps of: (a) providing a starting repertoire of nucleic acids encoding a variable domain that either includes a CDR3 to be replaced or lacks a CDR3 encoding region; (b) combining the repertoire with a donor nucleic acid encoding an amino acid sequence substantially as set out in SEQ ID NO:8, SEQ ID NO:26, SEQ ID NO:44, or SEQ ID NO:62 such that the donor nucleic acid is inserted into the CDR3 region in the repertoire, so as to provide a product repertoire of nucleic acids encoding a variable domain; (c) expressing the nucleic acids of the product repertoire; (d) selecting an antigen-binding fragment specific for SLPI; and (e) recovering the specific antigen-binding fragment or nucleic acid encoding the binding fragment.

28. An antibody produced by the method of claim 28.


SEQUENCE LISTING (112 sequences)

SEQ ID NO: 1 - DNA, 381 bp, Homo sapiens
  CDS (1)..(381)
caggtgcagctggtggagtctgggggaggcgtggtccagcctgggaggtccctgagactctcctgtgcagcgtctggattcaccttcagtacctatggcatgcactgggtccgccaggctccaggcaaggggctggagtgggtgacatttatatggtatgatggacgtaataaatactatgcagactccgtgaagggccgattcaccgtctccagagacaattccaagaacacgctgtatctgcaaatgaacagcctgagagccgaggacacggctgtgtattactgtgcgagaggggatgactacggtgactaccacccccactactactactacggtttggacgtctggggccaagggaccacggtcaccgtctcctca

SEQ ID NO: 2 - protein, 127 aa, Homo sapiens
QVQLVESGGGVVQPGRSLRLSCAASGFTFSTYGMHWVRQAPGKGLEWVTFIWYDGRNKYYADSVKGRFTVSRDNSKNTLYLQMNSLRAEDTAVYYCARGDDYGDYHPHYYYYGLDVWGQGTTVTVSS

SEQ ID NO: 3 - protein, 30 aa, Homo sapiens
QVQLVESGGGVVQPGRSLRLSCAASGFTFS

SEQ ID NO: 4 - protein, 5 aa, Homo sapiens
TYGMH

SEQ ID NO: 5 - protein, 14 aa, Homo sapiens
WVRQAPGKGLEWVT

SEQ ID NO: 6 - protein, 17 aa, Homo sapiens
FIWYDGRNKYYADSVKG

SEQ ID NO: 7 - protein, 32 aa, Homo sapiens
RFTVSRDNSKNTLYLQMNSLRAEDTAVYYCAR

SEQ ID NO: 8 - protein, 18 aa, Homo sapiens
GDDYGDYHPHYYYYGLDV

SEQ ID NO: 9 - protein, 11 aa, Homo sapiens
WGQGTTVTVSS

SEQ ID NO: 10 - DNA, 336 bp, Homo sapiens
  CDS (1)..(336)
gatattgtgatgacccagactccactctctctgtccgtcacccctggacagccggcctccatctcctgcaagtctagtcagagcctcctggatagtgatggaaagacctatttgtattggtacctgcagaagccaggccagcctccacagctcctgatctatgaagtttccaaccggttctctggagtgccagataggttcagtggcagcgggtcagggacagatttcacactgaagatcagccgggtggaggctgaggatgttgcatattattactgcatgcaaagtatacagcttccgtggacgttcggccaagggaccaaggtggaaatcaaa

SEQ ID NO: 11 - protein, 112 aa, Homo sapiens
DIVMTQTPLSLSVTPGQPASISCKSSQSLLDSDGKTYLYWYLQKPGQPPQLLIYEVSNRFSGVPDRFSGSGSGTDFTLKISRVEAEDVAYYYCMQSIQLPWTFGQGTKVEIK

SEQ ID NO: 12 - protein, 23 aa, Homo sapiens
DIVMTQTPLSLSVTPGQPASISC

SEQ ID NO: 13 - protein, 16 aa, Homo sapiens
KSSQSLLDSDGKTYLY

SEQ ID NO: 14 - protein, 15 aa, Homo sapiens
WYLQKPGQPPQLLIY

SEQ ID NO: 15 - protein, 7 aa, Homo sapiens
EVSNRFS

SEQ ID NO: 16 - protein, 32 aa, Homo sapiens
GVPDRFSGSGSGTDFTLKISRVEAEDVAYYYC

SEQ ID NO: 17 - protein, 9 aa, Homo sapiens
MQSIQLPWT

SEQ ID NO: 18 - protein, 10 aa, Homo sapiens
FGQGTKVEIK

SEQ ID NO: 19 - DNA, 366 bp, Homo sapiens
  CDS (1)..(366)
caggtgcagctgcaggagtcgggcccaggactggtgaagccttcacagaccctgtccctcacctgcactgtctctggtgtctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgttttactgtgcgagagagggggagtactacgatgagggggggtttgactactggggccagggaaccctggtcaccgtctcctca

SEQ ID NO: 20 - protein, 122 aa, Homo sapiens
QVQLQESGPGLVKPSQTLSLTCTVSGVSISSGGYYWSWIRQHPGKGLEWIGYIYYSGSTYYNPSLKSRVTISVDTSKNQFSLKLSSVTAADTAVFYCAREGEYYDEGGFDYWGQGTLVTVSS

SEQ ID NO: 21 - protein, 30 aa, Homo sapiens
QVQLQESGPGLVKPSQTLSLTCTVSGVSIS

SEQ ID NO: 22 - protein, 7 aa, Homo sapiens
SGGYYWS

SEQ ID NO: 23 - protein, 14 aa, Homo sapiens
WIRQHPGKGLEWIG

SEQ ID NO: 24 - protein, 16 aa, Homo sapiens
YIYYSGSTYYNPSLKS

SEQ ID NO: 25 - protein, 32 aa, Homo sapiens
RVTISVDTSKNQFSLKLSSVTAADTAVFYCAR

SEQ ID NO: 26 - protein, 12 aa, Homo sapiens
EGEYYDEGGFDY

SEQ ID NO: 27 - protein, 11 aa, Homo sapiens
WGQGTLVTVSS

SEQ ID NO: 28 - DNA, 324 bp, Homo sapiens
  CDS (1)..(324)
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctccgtggacgttcggccaagggaccaaggtggaaatcaaa

SEQ ID NO: 29 - protein, 108 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWPPWTFGQGTKVEIK

SEQ ID NO: 30 - protein, 23 aa, Homo sapiens
EIVMTQSPATLSVSPGERATLSC

SEQ ID NO: 31 - protein, 11 aa, Homo sapiens
RASQSVSSNLA

SEQ ID NO: 32 - protein, 15 aa, Homo sapiens
WYQQKPGQAPRLLIY

SEQ ID NO: 33 - protein, 7 aa, Homo sapiens
GASTRAT

SEQ ID NO: 34 - protein, 32 aa, Homo sapiens
GIPARFSGSGSGTEFTLTISSLQSEDFAVYYC

SEQ ID NO: 35 - protein, 10 aa, Homo sapiens [sequence omitted]

SEQ ID NO: 36 - protein, 10 aa, Homo sapiens
FGQGTKVEIK

SEQ ID NO: 37 - DNA, 381 bp, Homo sapiens
  CDS (1)..(381)
caggtgcagctggtggagtctgggggaggcgtggtccagcctgggaggtccctgagactctcctgtgcagcgtctggattcaccttcagtggctatggcatgcactgggtccgccaggctccaggcaaggggctggagtgggtggcatttatatggtataatggaaatgataattactatacagactccgtgaagggccgattcaccatctccagagacaattccaagaacacgctgtatctgcaaatgaacagcctgagagccgaggacacggctgtgtattactgtgcgagaggggatgactacggtgactaccacccccactactactactacggtatggacgtctggggccaagggaccacggtcaccgtctcctca

SEQ ID NO: 38 - protein, 127 aa, Homo sapiens
QVQLVESGGGVVQPGRSLRLSCAASGFTFSGYGMHWVRQAPGKGLEWVAFIWYNGNDNYYTDSVKGRFTISRDNSKNTLYLQMNSLRAEDTAVYYCARGDDYGDYHPHYYYYGMDVWGQGTTVTVSS

SEQ ID NO: 39 - protein, 30 aa, Homo sapiens; identical to SEQ ID NO: 3

SEQ ID NO: 40 - protein, 5 aa, Homo sapiens
GYGMH

SEQ ID NO: 41 - protein, 14 aa, Homo sapiens
WVRQAPGKGLEWVA

SEQ ID NO: 42 - protein, 17 aa, Homo sapiens
FIWYNGNDNYYTDSVKG

SEQ ID NO: 43 - protein, 32 aa, Homo sapiens
RFTISRDNSKNTLYLQMNSLRAEDTAVYYCAR

SEQ ID NO: 44 - protein, 18 aa, Homo sapiens
GDDYGDYHPHYYYYGMDV

SEQ ID NO: 45 - protein, 11 aa, Homo sapiens
WGQGTTVTVSS

SEQ ID NO: 46 - DNA, 336 bp, Homo sapiens
  CDS (1)..(336)
gatattgtgatgacccagactccactctctctgtccgtcacccctggacagccggcctccatctcctgcaagtctagtcagagcctcctggatagtgatggaaagacctatttgtattggtacctgcagaagccaggccagcctccacagctcctgatctatgaagtttccaaccggttctctggagtgccagataggttcagtggcagcgggtcagggacagatttcacactgaaaatcagccgggtggaggctgaggatattggggtttattactgcttgcaaagtatacagcttccgtggacgttcggccaagggaccaaggtggaaatcaaa

SEQ ID NO: 47 - protein, 112 aa, Homo sapiens
DIVMTQTPLSLSVTPGQPASISCKSSQSLLDSDGKTYLYWYLQKPGQPPQLLIYEVSNRFSGVPDRFSGSGSGTDFTLKISRVEAEDIGVYYCLQSIQLPWTFGQGTKVEIK

SEQ ID NO: 48 - protein, 23 aa, Homo sapiens
DIVMTQTPLSLSVTPGQPASISC

SEQ ID NO: 49 - protein, 16 aa, Homo sapiens
KSSQSLLDSDGKTYLY

SEQ ID NO: 50 - protein, 15 aa, Homo sapiens
WYLQKPGQPPQLLIY

SEQ ID NO: 51 - protein, 7 aa, Homo sapiens
EVSNRFS

SEQ ID NO: 52 - protein, 32 aa, Homo sapiens
GVPDRFSGSGSGTDFTLKISRVEAEDIGVYYC

SEQ ID NO: 53 - protein, 9 aa, Homo sapiens
LQSIQLPWT

SEQ ID NO: 54 - protein, 10 aa, Homo sapiens
FGQGTKVEIK

SEQ ID NO: 55 - DNA, 366 bp, Homo sapiens
  CDS (1)..(366)
caggtgcagctgcaggagtcgggcccaggactggtgaagccttcacagaccctgtccctcacctgcactgtctctggtggctccatcaaaagtggtgattactactggagctgggtccgccagctcccagggaagggcctggagtggattggatacatctattacagtgggagcacctactacaacccgtccctcaagagtcgagttaccatatcaatagacacgtctaagaaccagttctccctgaagctgagctctgtgactgccgcggacacggccgtgtattactgtgcgagagaagggtcttactatgattcgggaggctttgactactggggccagggaaccctggtcaccgtctcctca

SEQ ID NO: 56 - protein, 122 aa, Homo sapiens
QVQLQESGPGLVKPSQTLSLTCTVSGGSIKSGDYYWSWVRQLPGKGLEWIGYIYYSGSTYYNPSLKSRVTISIDTSKNQFSLKLSSVTAADTAVYYCAREGSYYDSGGFDYWGQGTLVTVSS

SEQ ID NO: 57 - protein, 30 aa, Homo sapiens
QVQLQESGPGLVKPSQTLSLTCTVSGGSIK

SEQ ID NO: 58 - protein, 7 aa, Homo sapiens
SGDYYWS

SEQ ID NO: 59 - protein, 14 aa, Homo sapiens
WVRQLPGKGLEWIG

SEQ ID NO: 60 - protein, 16 aa, Homo sapiens
YIYYSGSTYYNPSLKS

SEQ ID NO: 61 - protein, 32 aa, Homo sapiens
RVTISIDTSKNQFSLKLSSVTAADTAVYYCAR

SEQ ID NO: 62 - protein, 12 aa, Homo sapiens
EGSYYDSGGFDY

SEQ ID NO: 63 - protein, 11 aa, Homo sapiens
WGQGTLVTVSS

SEQ ID NO: 64 - DNA, 324 bp, Homo sapiens
  CDS (1)..(324)
gaaatagtgatgacgcagtctccaaccaccctgtctgtgtctccaggggaaagagccaccctctcctgtagggccggtcagagtgttggcaacaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctccgtggacgttcggccaagggaccaaggtggaaatcaac

SEQ ID NO: 65 - protein, 108 aa, Homo sapiens
EIVMTQSPTTLSVSPGERATLSCRAGQSVGNNLAWYQQKPGQAPRLLIYGASTRATGIPARFSGSGSGTEFTLTISSLQSEDFAVYYCQQYNNWPPWTFGQGTKVEIN

SEQ ID NO: 66 - protein, 23 aa, Homo sapiens
EIVMTQSPTTLSVSPGERATLSC

SEQ ID NO: 67 - protein, 11 aa, Homo sapiens
RAGQSVGNNLA

SEQ ID NO: 68 - protein, 15 aa, Homo sapiens
WYQQKPGQAPRLLIY

SEQ ID NO: 69 - protein, 7 aa, Homo sapiens
GASTRAT

SEQ ID NO: 70 - protein, 32 aa, Homo sapiens; identical to SEQ ID NO: 34

SEQ ID NO: 71 - protein, 10 aa, Homo sapiens [sequence omitted]

SEQ ID NO: 72 - protein, 10 aa, Homo sapiens
FGQGTKVEIN

SEQ ID NO: 73 - protein, 122 aa, Homo sapiens
  misc_feature (27)..(27): Wherein Xaa is Gly or Val
QVQLQESGPGLVKPSQTLSLTCTVSGXSIXSGXYYWSWXRQXPGKGLEWIGYIYYSGSTYYNPSLKSRVTISXDTSKNQFSLKLSSVTAADTAVXYCAREGXYYDXGGFDYWGQGTLVTVSS

SEQ ID NO: 74 - protein, 127 aa, Homo sapiens
  misc_feature (31)..(31): Wherein Xaa is Ser, Thr or Gly
QVQLVESGGGVVQPGRSLRLSCAASGFTFSXYGMHWVRQAPGKGLEWVXXIWYXGXXXYYXDSVKGRFTXSRDNSKNTLYLQMNSLRAEDTAVYYCARGDDYGDYHPHYYYYGXDVWGQGTTVTVSS

SEQ ID NO: 75 - protein, 108 aa, Homo sapiens
  misc_feature (9)..(9): Wherein Xaa is Ala or Thr
EIVMTQSPXTLSVSPGERATLSCRAXQSVXXNLAWYQQKPGQAPRLLIYGASTRATGIPARFSGSGSGTEFTLTISSLQSEDFAVYYCQQYNNWPPWTFGQGTKVEIX

SEQ ID NO: 76 - protein, 112 aa, Homo sapiens
  misc_feature (31)..(31): Wherein Xaa is His or Asp
DIVMTQTPLSLSVTPGQPASISCKSSQSLLXSDGKTYLYWYLQKPGQPPQLLIYEVSNRFSGVPDRFSGSGSGTDFTLKISRVEAEDXXXYYCXQSIQLPWTFGQGTKVEIK

SEQ ID NO: 77 - protein, 7 aa, Homo sapiens
SGGYYWS

SEQ ID NO: 78 - protein, 5 aa, Homo sapiens
SYGMH

SEQ ID NO: 79 - protein, 17 aa, Homo sapiens
VIWYDGSNKYYADSVKG

SEQ ID NO: 80 - protein, 16 aa, Homo sapiens
KSSQSLLHSDGKTYLY

SEQ ID NO: 81 - DNA, 299 bp, Homo sapiens
  CDS (1)..(297)
caggtgcagctgcaggagtcgggcccaggactggtgaagccttcacagaccctgtccctcacctgtactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagaga

SEQ ID NO: 82 - protein, 99 aa, Homo sapiens
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCAR

SEQ ID NO: 83 - DNA, 296 bp, Homo sapiens
  CDS (1)..(294)
caggtacagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgcgaagggccgattcaccatctccagagacaattccacgaacacgctgtttctgcaaatgaacagcctgagagccgaggacacggctgtgtattactgtgcgagaga

SEQ ID NO: 84 - protein, 98 aa, Homo sapiens
QVQLVESGGGVVQPGRSLRLSCAASGFTFSSYGMHWVRQAPGKGLEWVAVIWYDGSNKYYADSAKGRFTISRDNSTNTLFLQMNSLRAEDTAVYYCAR

SEQ ID NO: 85 - DNA, 31 bp, Homo sapiens
aggatattgtagtagtaccagctgctatacc

SEQ ID NO: 86 - protein, 8 aa, Homo sapiens
RILYQLLY

SEQ ID NO: 87 - protein, 10 aa, Homo sapiens
GYCSSTSCYT

SEQ ID NO: 88 - protein, 9 aa, Homo sapiens
DIVVVPAAI

SEQ ID NO: 89 - DNA, 31 bp, Homo sapiens
gtattactatggttcggggagttattataac

SEQ ID NO: 90 - protein, 9 aa, Homo sapiens
VLLWFGELL

SEQ ID NO: 91 - protein, 10 aa, Homo sapiens
YYYGSGSYYN

SEQ ID NO: 92 - protein, 9 aa, Homo sapiens
ITMVRGVII

SEQ ID NO: 93 - DNA, 16 bp, Homo sapiens
tgactacggtgactac

SEQ ID NO: 94 - protein, 3 aa, Homo sapiens
LRL

SEQ ID NO: 95 - protein, 5 aa, Homo sapiens
DYGDY

SEQ ID NO: 96 - protein, 4 aa, Homo sapiens
TTVT

SEQ ID NO: 97 - DNA, 37 bp, Homo sapiens
gtattatgattacgtttgggggagttatcgttatacc

SEQ ID NO: 98 - protein, 11 aa, Homo sapiens
VLLRLGELSLY

SEQ ID NO: 99 - protein, 12 aa, Homo sapiens
YYDYVWGSYRYT

SEQ ID NO: 100 - protein, 11 aa, Homo sapiens
IMITFGGVIVI

SEQ ID NO: 101 - DNA, 48 bp, Homo sapiens
  CDS (3)..(47)
actactttgactactggggccagggaaccctggtcaccgtctcctcag

SEQ ID NO: 102 - protein, 15 aa, Homo sapiens
YFDYWGQGTLVTVSS

SEQ ID NO: 103 - DNA, 63 bp, Homo sapiens
  CDS (3)..(62)
attactactactactacggtatggacgtctggggccaagggaccacggtcaccgtctcctcag

SEQ ID NO: 104 - protein, 20 aa, Homo sapiens
YYYYYGMDVWGQGTTVTVSS

SEQ ID NO: 105 - DNA, 287 bp, Homo sapiens
  CDS (1)..(285)
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctcc

SEQ ID NO: 106 - protein, 95 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 107 - DNA, 302 bp, Homo sapiens
  CDS (1)..(300)
gatattgtgatgacccagactccactctctctgtccgtcacccctggacagccggcctccatctcctgcaagtctagtcagagcctcctgcatagtgatggaaagacctatttgtattggtacctgcagaagccaggccagtctccacagctcctgatctatgaagtttccaaccggttctctggagtgccagataggttcagtggcagcgggtcagggacagatttcacactgaaaatcagccgggtggaggctgaggatgttggggtttattactgcatgcaaagtatacagcttcctcc

SEQ ID NO: 108 - protein, 100 aa, Homo sapiens
DIVMTQTPLSLSVTPGQPASISCKSSQSLLHSDGKTYLYWYLQKPGQSPQLLIYEVSNRFSGVPDRFSGSGSGTDFTLKISRVEAEDVGVYYCMQSIQLP

SEQ ID NO: 109 - DNA, 38 bp, Homo sapiens
  CDS (2)..(37)
gtggacgttcggccaagggaccaaggtggaaatcaaac

SEQ ID NO: 110 - protein, 12 aa, Homo sapiens
WTFGQGTKVEIK

SEQ ID NO: 111 - DNA, 594 bp, Homo sapiens
  CDS (19)..(414)
gtcactcctgccttcaccatgaagtccagcggcctcttccccttcctggtgctgcttgccctgggaactctggcaccttgggctgtggaaggctctggaaagtccttcaaagctggagtctgtcctcctaagaaatctgcccagtgccttagatacaagaaacctgagtgccagagtgactggcagtgtccagggaagaagagatgttgtcctgacacttgtggcatcaaatgcctggatcctgttgacaccccaaacccaacaaggaggaagcctgggaagtgcccagtgacttatggccaatgtttgatgcttaacccccccaatttctgtgagatggatggccagtgcaagcgtgacttgaagtgttgcatgggcatgtgtgggaaatcctgcgtttcccctgtgaaagcttgattcctgccatatggaggaggctctggagtcctgctctgtgtggtccaggtcctttccaccctgagacttggctccaccactgatatcctcctttggggaaaggcttggcacacagcaggctttcaagaagtgccagttgatcaatgaataaataaacgagcctatttctctttgcac

SEQ ID NO: 112 - protein, 132 aa, Homo sapiens
MKSSGLFPFLVLLALGTLAPWAVEGSGKSFKAGVCPPKKSAQCLRYKKPECQSDWQCPGKKRCCPDTCGIKCLDPVDTPNPTRRKPGKCPVTYGQCLMLNPPNFCEMDGQCKRDLKCCMGMCGKSCVSPVKA